(19) United States
(12) Patent Application Publication
(10) Pub. No.: US 2026/0016482 A1
(43) Pub. Date: Jan. 15, 2026

(54) METHODS FOR ANALYZING BIOLOGICAL SAMPLES

(71) Applicant: Seer, Inc., Redwood City, CA (US)

(72) Inventor: Iman MOHTASHEMI, Mountain House, CA (US)

(21) Appl. No.: 18/881,691

(22) PCT Filed: Jul. 7, 2023
(86) PCT No.: PCT/US2023/069793

§ 371 Date: Jan. 6, 2025

Related U.S. Application Data
(60) Provisional application No. 63/368,028, filed on Jul. 8, 2022.
(60) Provisional application No. 63/368,474, filed on Jul. 14, 2022.
(60) Provisional application No. 63/386,483, filed on Dec. 7, 2022.

(51) Int. Cl. G01N 33/68; G01N 27/623; G01N 27/624; G01N 30/02; G01N 30/72; G01N 30/88; H01J 49/00

(52) U.S. Cl. CPC G01N 33/6848; G01N 27/623; G01N 27/624; G01N 30/7233; G01N 30/88; H01J 49/004; G01N 2030/027; G01N 2030/8813

(57) ABSTRACT

Described herein are methods for analyzing biological samples by liquid chromatography-tandem mass spectrometry or direct injection mass spectrometry.

### CROSS-REFERENCE

This application claims the benefit of U.S. Provisional Application No. 63/368,028, filed Jul. 8, 2022, U.S. Provisional Application No. 63/368,474, filed Jul. 14, 2022, and U.S. Provisional Application No. 63/386,483, filed Dec. 7, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jun. 28, 2023, is named 53344-764_601_SL.xml and is 4,529 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Nanoflow liquid chromatography-tandem mass spectrometry (nLC-MS/MS) has been of interest to proteomics due to its high sensitivity and throughput. LC systems can be coupled to MS systems to minimize ionization suppression, as ESI can pose a major bottleneck of MS-based detection schemes. Comprehensive proteome coverage and throughput are inversely correlated, and scaling proteomics (as defined the number of samples analyzed with no compromise in coverage) remains a challenge.

### SUMMARY

Disclosed herein are methods for solving the inherent problem of nLC-MS/MS with scaling proteomics by removing the chromatography domain and using surfaces (e.g. particles) for sample complexity reduction. The methods disclosed herein may, in some embodiments, increase throughput by several orders of magnitude without compromising identification rates.

In some aspects, the present disclosure provides a method comprising: contacting a plurality of biomolecules with a surface to adsorb the plurality of biomolecules on the surface; and performing mass spectrometry on the plurality of biomolecules to generate a mass spectrum, wherein the mass spectrometry is (i) performed without chromatographic separation or (ii) performed using chromatographic separation with a gradient length of at most 30 minutes.

In some embodiments, the gradient length is at most 25, 20, 15, 10, 9, 8, 7, 6, 5, 4, 3, 2, or 1 minute.

In some embodiments, the chromatographic separation uses a chromatography column comprising a length of at most 30, 25, 20, 15, 10, 5, 4, 3, 2, 1, 0.9, 0.8, 0.7, 0.6, 0.5, 0.4, 0.3, 0.2, or 0.1 centimeters.

In some embodiments, the performing the mass spectrometry comprises performing ion mobility separation.

In some embodiments, the ion mobility separation is performed using travelling-wave ion mobility separation.

In some embodiments, the travelling-wave ion mobility separation is performed along a distance greater than 5 m or greater than 10 m.

In some embodiments, the travelling-wave ion mobility separation is performed along a serpentine path.

In some embodiments, the ion mobility separation is performed using high field asymmetric waveform ion mobility spectrometry (FAIMS).

In some embodiments, the performing the mass spectrometry comprises performing direct infusion mass spectrometry.

In some embodiments, the direct infusion mass spectrometry is performed in less than about 10 minutes, less than about 5 minutes, less than about 3 minutes, or less than about 2 minutes.

In some embodiments, the mass spectrometry is performed with an isolation width of about 0.4, 0.6, 0.8, 1, or 2 m/z.

In some embodiments, the mass spectrometry is performed with a maximum injection time of about 500 ms, 1 s, 5 s, 30 s, 1 min, 2 min, 3 min, 4 min, 5 min, or 10 min.

In some embodiments, the mass spectrometry is performed with a resolution of about 120, 240, or 500 K.

In some embodiments, the mass spectrometry is performed with a resolution of about 30K to about 800K. In some embodiments, the contacting the plurality of biomolecules with the surface to adsorb the plurality of biomolecules on the surface comprises contacting a biological sample with the surface.

In some embodiments, the biological sample comprises blood, plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample comprises blood, serum, or plasma.

In some embodiments, the biological sample is a biofluid.

In some embodiments, the biological sample comprises cell lysate.

In some embodiments, the surface is a particle.

In some embodiments, the contacting the plurality of biomolecules with the surface to adsorb the plurality of biomolecules on the surface comprises incubating the biomolecules with the surface for at least 15 minutes.

In some embodiments, the incubating is performed at a temperature of about 5° C. to about 40° C.

In some embodiments, the mass spectrometry comprises tandem mass spectrometry.

In some embodiments, the mass spectrometry comprises Fourier Transform mass spectrometry.

In some embodiments, the mass spectrometry comprises time-of-flight mass spectrometry.

In some embodiments, performing mass spectrometry identifies at least about 300 protein groups.

In some embodiments, performing mass spectrometry comprises data independent acquisition (DIA).

In some embodiments, a surface comprises a plurality of particles having a polydispersity index of greater than 0.5, greater than 0.75, or greater than 1.0.

In some embodiments, a surface comprises a plurality of particles having a polydispersity index of less than 0.1.

In some embodiments, a plurality of biomolecules comprise a plurality of proteins.

In some embodiments, a surface selectively adsorbs extracellular vesicles.

In some embodiments, a surface is ammonium-functionalized.

In some embodiments, a surface comprises polyethyleneimine.

In some embodiments, a surface comprises crosslinked polyethyleneimine.

In some embodiments, a surface comprises polyethylene oxide and an aromatic group.

In some aspects, the present disclosure provides a method comprising: contacting a plurality of biomolecules with a surface to adsorb the plurality of biomolecules on the surface; performing mass spectrometry on the plurality of biomolecules to generate a multiplexed mass spectrum; and deconvoluting the multiplexed mass spectrum to generate a plurality of deconvoluted MS signals, wherein the deconvoluting is based on a type of the surface.

In some embodiments, the deconvoluting is based on genomic information associated with the plurality of biomolecules.

In some embodiments, the genomic information comprises a spectral library of a plurality of expressible polyamino acids by an organism.

In some embodiments, the spectral library comprises a plurality of mass spectral signals of the plurality of expressible polyamino acids.

In some embodiments, the performing the mass spectrometry comprises scanning the plurality of biomolecules across a plurality of mass windows.

In some embodiments, the scanning is performed at a frequency between 20 and 80 Hertz.

In some embodiments, the plurality of mass windows is between 400 and 1600 Daltons.

In some embodiments, the plurality of mass windows comprises a window of about 2 Daltons.

In some embodiments, the multiplexed mass spectrum comprises signals within the window.

In some embodiments, the plurality of mass spectral signals in the spectral library is within the window.

In some embodiments, the deconvoluting is based on a spectral library of a subset of the plurality of expressible polyamino acids, wherein the subset is configured to be adsorbed preferentially to the surface compared to an expressible polyamino acid not in the subset.

In some embodiments, the plurality of biomolecules is less abundant compared to the most abundant biomolecule in a sample by at most about 6 orders of magnitude.

In some embodiments, the plurality of biomolecules is less abundant compared to the most abundant biomolecule in a sample by at least about 5 orders of magnitude and at most about 8 orders of magnitude.

In some embodiments, the plurality of biomolecules is less abundant compared to the most abundant biomolecule in a sample by at least about 7 orders of magnitude.

In some embodiments, the contacting the plurality of biomolecules with the surface to adsorb the plurality of biomolecules on the surface comprises contacting a biological sample with the surface.

In some embodiments, the biological sample comprises blood, plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample comprises blood, serum, or plasma.

In some embodiments, the biological sample comprises cell lysate.

In some embodiments, the biological sample is a biofluid.

In some embodiments, the surface is a particle.

In some embodiments, the contacting the plurality of biomolecules with the surface to adsorb the plurality of biomolecules on the surface comprises incubating the biomolecules with the surface for at least 15 minutes.

In some embodiments, the incubating is performed at a temperature of about 5° C. to about 40° C.

In some embodiments, the mass spectrometry comprises tandem mass spectrometry.

In some embodiments, the mass spectrometry comprises Fourier Transform mass spectrometry.

In some embodiments, the mass spectrometry comprises time-of-flight mass spectrometry.

In some embodiments, the mass spectrometry comprises data independent acquisition (DIA).

In some embodiments, the mass spectrometry comprises a targeted mass spectrometry.

In some embodiments, the targeted mass spectrometry comprises multiple reaction monitoring (MRM).

In some embodiments, the surface selectively adsorbs extracellular vesicles.

In some embodiments, the surface is ammonium-functionalized.

In some embodiments, the surface comprises polyethyleneimine.

In some embodiments, the surface comprises crosslinked polyethyleneimine.

In some embodiments, the surface comprises polyethylene oxide and an aromatic group.

Some embodiments disclosed herein include a method comprising incubating a biological sample with one or more surfaces to form a biomolecule corona on the one or more surfaces; performing mass spectrometry on biomolecules from the biomolecule corona to generate a mass spectrum, wherein the mass spectrometry is (i) performed without chromatographic separation or (ii) performed using chromatographic separation with a gradient length of at most 10 minutes.

In some embodiments, the gradient length is at most 9, 8, 7, 6, 5, 4, 3, 2, or 1 minute.

In some embodiments, the chromatographic separation uses a chromatography column comprising a length of at most 30, 25, 20, 15, 10, 5, 4, 3, 2, 1, 0.9, 0.8, 0.7, 0.6, 0.5, 0.4, 0.3, 0.2, or 0.1 centimeters.

In some embodiments, the performing the mass spectrometry comprises performing ion mobility separation.

In some embodiments, wherein the ion mobility separation is performed using high field asymmetric waveform ion mobility spectrometry (FAIMS).

In some embodiments, the ion mobility separation is performed using travelling-wave ion mobility separation.

In some embodiments, the travelling-wave ion mobility separation is performed along a distance greater than 5 m or greater than 10 m.

In some embodiments, the travelling-wave ion mobility separation is performed along a serpentine path.

In some embodiments, the performing mass spectrometry comprises performing direct infusion mass spectrometry.

In some embodiments, the performing mass spectrometry is without chromatographic separation.

In some embodiments, the performing mass spectrometry comprises tandem mass spectrometry.

In some embodiments, the performing mass spectrometry comprises data independent acquisition (DIA).

In some embodiments, the performing mass spectrometry comprises a targeted mass spectrometry.

In some embodiments, the targeted mass spectrometry comprises multiple reaction monitoring (MRM).

In some embodiments, the performing mass spectrometry comprises electrospray injection.

In some embodiments, the biomolecules from the biomolecule corona comprise proteins.

In some embodiments, the performing mass spectrometry comprises detecting at least 50, 100, 200, 250, 300, 400, 500, or 1000 proteins in the biological sample.

In some embodiments, the performing mass spectrometry comprises detecting at least 50, 100, 200, 250, 300, 400, 500, or 1000 protein groups in the biological sample.

In some embodiments, the method further comprises, before (b), removing biomolecules from the biomolecule corona.

In some embodiments, the removing biomolecules from biomolecule corona comprises digesting the biomolecule corona with an enzyme.

In some embodiments, the enzyme is a protease, such as trypsin.

In some embodiments, the method further comprises, before (b), alkylating and reducing the biomolecules from the biomolecule corona.

In some embodiments, the method further comprises, before (b), alkylating, reducing, and digesting the biomolecules from the biomolecule corona.

In some embodiments, the method further comprises before (b), apply one or more washing steps to the biomolecules from the biomolecule corona.

In some embodiments, the incubating comprises maintaining the biological sample at a temperature of 20° C. to 50° C. for at least 15 minutes.

In some embodiments, the biological sample comprises blood, plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample comprises blood, serum, or plasma.

In some embodiments, the biological sample comprises cell lysate.

In some embodiments, the one or more surfaces comprise a particle.

In some embodiments, the particle is a porous particle.

In some embodiments, the particle is a microparticle.

In some embodiments, the particle is nanoparticle.

In some embodiments, the particle comprises a paramagnetic material.

In some embodiments, the paramagnetic material is a superparamagnetic material.

In some embodiments, the paramagnetic material comprises iron oxide

In some embodiments, the biological sample is incubated with a first surface and a second surface having different physicochemical properties such that different protein coronas are formed on each surface.

In some embodiments, the first surface and the second surface comprise a different sign of charge.

In some embodiments, the first surface and the second surface comprise a different sign of zeta potential.

In some embodiments, the first surface and the second surface comprise the same sign of charge.

In some embodiments, the first surface and the second surface comprise the same sign of zeta potential.

In some embodiments, the first surface, the second surface, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof.

In some embodiments, the first surface and the second surface are incubated in separate aliquots of the biological sample.

In some embodiments, the mass spectrometry is performed separately for the biomolecule corona from the first surface and the biomolecule corona from the second surface.

In some embodiments, the first surface and the second surface are incubated in a single volume of the biological sample.

In some embodiments, the method further comprises, before (b), washing the biomolecule corona with a wash composition.

In some embodiments, the method further comprises, determining a biological state of a subject using the mass spectrum.

In some embodiments, the biological sample is incubated with at least 2, 3, 4, 5, 6, 7, 8, 9, or 10 surfaces having different physicochemical properties.

In some embodiments, the biological sample is incubated with 3 to 8 surfaces having different physicochemical properties.

In some embodiments, the performing mass spectrometry comprises at least 2, 3, 4, 5, 6, 7, 8, 9, or 10 separate injections.

In some embodiments, the performing mass spectrometry comprises 3-8 separate injections.

In some embodiments, each of the separate injections correspond to biomolecule corona obtained from different surfaces.

In some embodiments, at least one of the surfaces selectively adsorbs extracellular vesicles.

In some embodiments, at least one of the surfaces is ammonium-functionalized.

In some embodiments, at least one of the surfaces comprise polyethyleneimine.

In some embodiments, at least one of the surfaces comprise crosslinked polyethyleneimine.

In some embodiments, at least one of the surfaces comprises polyethylene oxide and an aromatic group.

Some embodiments disclosed herein include a method comprising incubating a biological sample with a first magnetic particle to form a first protein corona; incubating the biological sample with a second magnetic particle to form a second protein corona, wherein the first magnetic particle and the second magnetic particle have different physicochemical properties, and wherein the first magnetic particle and the second magnetic particle are incubated with separate volumes of the biological sample; and performing mass spectrometry using direct injection on proteins from the first protein corona and the second protein corona to generate a mass spectrum, wherein separate injections are performed for the first protein corona and the second protein corona.

In some embodiments, wherein at least 500 different protein groups are detected.

In some embodiments, at least 1000 different protein groups are detected.

In some embodiments, the biological sample is blood, serum, or plasma.

In some embodiments, the first magnetic particle and the second magnetic particle have a different sign of zeta potential.

In some embodiments, an outer surface of the first magnetic particle comprises an amine functional group and an outer surface of the second magnetic particle comprises a carboxyl functional group.

In some embodiments, the first magnetic particle comprises a polymeric outer surface and the second magnetic particle comprises a non-polymeric outer surface.

In some embodiments, at least 50 unique protein groups are detected from the first protein corona that are not detected from the second protein corona

In some embodiments, at least 100 unique protein groups are detected from the first protein corona that are not detected from the second protein corona.

In some embodiments, the first magnetic particle and the second magnetic particle each have a polydispersity index less than 0.25.

In some embodiments, the first magnetic particle and the second magnetic particle each have a polydispersity index less than 0.1.

In some embodiments, the first magnetic particle and the second magnetic particle are nanoparticles.

In some embodiments, the first magnetic particle and the second magnetic particle have a diameter less than 500 nm.

In some embodiments, the mass spectrometry is performed in less than 10 minutes.

### BRIEF DESCRIPTION OF THE DRAWINGS

This patent application contains at least one drawing executed in color. Copies of this patent or patent application with color drawing(s) will be provided by the Office upon request and payment of the necessary fee.

FIG. 1 is a chart illustrating the steps for determining protein identifications.

FIG. 2 shows a linear system of equations for a spectral demultiplexing algorithm.

FIG. 3 shows a direct infusion MS spectrum (relative intensity vs. m/z) of extracted four proteomes ((1) KQTALVELVK (SEQ ID NO: 1); (2) YTCLPGYVR (SEQ ID NO: 2); (3) ESDTSYVSLK (SEQ ID NO: 3); and (4) DCHLAQVPSHTVVAR SEQ ID NO: 4)).

FIG. 4A shows a direct infusion MS spectrum (relative intensity vs. m/z) of KQTALVELVK (SEQ ID NO: 1).

FIG. 4B shows a direct infusion MS spectrum (relative intensity vs. m/z) of YTCLPGYVR (SEQ ID NO: 2).

FIG. 4C shows a direct infusion MS spectrum (relative intensity vs. m/z) of ESDTSYVSLK (SEQ ID NO: 3).

FIG. 4D shows a direct infusion MS spectrum (relative intensity vs. m/z) of DCHLAQVPSHTVVAR (SEQ ID NO: 4).

FIG. 5A shows a graph illustrating unique peptides found by nanoparticles at 60 k resolution.

FIG. 5B shows a graph illustrating unique peptides found by nanoparticles at 120 k resolution.

FIG. 6 shows a flow chart for proteomes analysis by using AlphaPept's “modified” protein inference module.

FIG. 7A shows a waterfall plot (Carr database) for MS-direct nanoparticle panel. (Number of database entries: 5304; number of input protein groups: 159; number of separated protein ID's: 159; and number of matches to database: 133)

FIG. 7B shows a waterfall plot (Carr database) for NEAT triplicates. (Number of database entries: 5304; number of input protein groups: 130; number of separated protein ID's: 130; and number of matches to database: 108)

FIG. 8 shows a bar graph illustrating the number of protein groups for CsoDIAq, matrix demultiplexing-Neat, matrix demultiplexing-Panel, and 30 min-Neat-LCMS.

FIGS. 9A-9C show the waterfall plots (Carr database) for a 3 nanoparticle panel.

FIG. 10 shows a design of experimental matrix in which sensitivity, resolution, and throughput are balanced. Experimental conditions are shown in Table 3.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Liquid chromatography-mass spectrometry (LC-MS) is an important analytical technique in proteomics where proteins are digested and separated by high performance chromatography and detected by mass spectrometers. Particularly, nanoflow liquid chromatography-tandem mass spectrometry (nLC-MS/MS) has been of interest to proteomics due to its high sensitivity and throughput. However, a large-scale analysis by nLC-MS/MS remains a challenge because of the complexity and dynamic range of proteomics samples, such as plasma or tissue lysates.

Two primary factors limiting the large scale of nLC-MS/MS can include electrospray (ESI) dynamic range and instrument duty cycle. Although instrument scan rates continue to improve, ESI has been a rate-limiting factor. Better peak capacity may directly contribute to identification rates. This means that decreasing the dynamic concentration range of a mixture at a given analytical time point for an MS detector is likely to contribute to the increased identification rate since ESI saturation occurs at five orders of magnitude of dynamic range. However, gradient length is a major contributor to peak capacity, and thus, the analyst can be forced to trade-off identification rate for throughput.

The existing method of reducing an intra-scan dynamic range in an LC-MS run is to increase the gradient length. Increasing the gradient length can increase the peak capacity, which can allow co-eluting peaks to be resolved, and can alleviate the ionization suppression in ESI, which in turn, can reduce throughput.

Also disclosed herein are methods for detecting biomolecules in a sample. The method may include incubating a biological sample with one or more surfaces to form a biomolecule corona on the one or more surfaces; performing mass spectrometry on biomolecules from the biomolecule corona to generate a mass spectrum, wherein the mass spectrometry is (i) performed without chromatographic separation or (ii) performed using chromatographic separation with a gradient length of at most 10 minutes.

Also disclosed herein are method comprising incubating a biological sample with a first magnetic particle to form a first protein corona; incubating the biological sample with a second magnetic particle to form a second protein corona, wherein the first magnetic particle and the second magnetic particle have different physicochemical properties, and wherein the first magnetic particle and the second magnetic particle are incubated with separate volumes of the biological sample; and performing mass spectrometry using direct injection on proteins from the first protein corona and the second protein corona to generate a mass spectrum, wherein separate injections are performed for the first protein corona and the second protein corona.

In some case, the methods disclosed herein are advantageous because they can significantly reduce the amount time required to perform mass spectrometry. A standard proteomic mass spectrometry analysis can require liquid chromatography gradients of one hour or more. The methods disclosed herein may be performed in less than 20 minutes, or even less than 10 minutes, while still detecting and/or quantifying a large number of biomolecules, such as at least 1000 proteins.

In some embodiments, disclosed herein is a method of reducing the dynamic range by using nanoparticles. A highly-parallel protein quantitation platform, integrating nanoparticle (NP) protein coronas with LC-MS, can provide efficient proteomic profiling because this enrichment is affinity-based but not concentration-dependent. At equilibrium, binding kinetics can determine the protein corona. Thus, in some embodiments, disclosed herein is a method of compressing a large plasma dynamic range, such as from about 109 to about 1012, to five orders by using a panel of two, three, four, five or more different nanoparticles with different physicochemical properties on their surfaces.

In some embodiments, disclosed herein is a method of designing a panel of a plurality of nanoparticles to leverage the nanoparticle affinity where the plurality of nanoparticles are designed to separately enrich proteins at different concentration ranges. In some embodiments, the method of designing a panel of a plurality of nanoparticles includes: building a training model for protein corona formation; tuning particle chemistry to enrich for proteins at different concentrations; injecting each nanoparticle-enriched peptide pool for about 30 seconds, about 35 seconds, about 40 seconds, 45 seconds, about 50 seconds, or about 55 seconds of MS detection; building predicted spectral library using the reference genome; and deconvoluting multiplexed spectra by using linear algebra.

In some embodiments, disclosed herein is a method for performing mass spectrometry. In some embodiments, the method comprises fractionating a sample with one or more nanoparticles. In some embodiments, the method comprises performing ion mobility separation on a plurality of biomolecules. For example, the ion mobility separation may comprise travelling wave ion mobility or high field asymmetric waveform ion mobility spectrometry. In some embodiments, the travelling wave ion mobility comprises a separation distance of at least 5 m or 10 m. In some embodiments, the travelling wave ion mobility comprises a serpentine path. In some embodiments, the method comprises performing direct infusion mass spectrometry. In some embodiments, the method comprises performing a demultiplexing algorithm. In some embodiments, a quadruple transmission can be performed with a mass range of 400-1200, a 4 Dalton window at 3 CVs scanning at 25 Hertz, which provides about 24 seconds per injection.

In some embodiments, suitable nanoparticles include, but are not limited to, for example, organic nanoparticles, non-organic particles, inorganic nanoparticles, or combinations thereof. In some embodiments the nanoparticles are, for example, micelles, extracellular vesicles, liposomes, iron oxide, graphene, silica, protein-based particles, polystyrene, silver, and gold particles, quantum dots, palladium, platinum, titanium, and combinations thereof. In some embodiments, nanoparticles are polymeric nanoparticles. In some embodiments, nanoparticles are metal oxides. In some embodiments, nanoparticles are metals. In some embodiments, nanoparticles are ceramics. In some embodiments, nanoparticles are liposomes. One skilled in the art would be able to select and prepare suitable nanoparticles. In some embodiments, suitable nanoparticles are less than 1000 nm in at least one direction. In some embodiments, the nanoparticles are less than about 100 nm in at least one direction.

In some embodiments, a nanoparticle is a particle that is smaller than about 1000 nm in diameter. In some embodiments, a nanoparticle is a particle that is smaller than about 900 nm in diameter. In some embodiments, a nanoparticle is a particle that is smaller than about 800 nm in diameter. In some embodiments, a nanoparticle is a particle that is smaller than about 700 nm in diameter. In some embodiments, a nanoparticle is a particle that is smaller than about 600 nm in diameter. In some embodiments, a nanoparticle is a particle that is smaller than about 500 nm in diameter. In some embodiments, a nanoparticle is a particle that is smaller than about 400 nm in diameter. In some embodiments, a nanoparticle is a particle that is smaller than about 300 nm in diameter. In some embodiments, a nanoparticle is a particle that is smaller than about 200 nm in diameter. In some embodiments, a nanoparticle is a particle that is smaller than about 100 nm in diameter.

In some embodiments, the size of nanoparticle ranges from about 1 nm to about 1,000 nm. In some embodiments, the size of nanoparticle ranges from about 1 nm to about 10 nm, about 1 nm to about 50 nm, about 1 nm to about 100 nm, about 1 nm to about 200 nm, about 1 nm to about 300 nm, about 1 nm to about 500 nm, about 1 nm to about 600 nm, about 1 nm to about 700 nm, about 1 nm to about 800 nm, about 1 nm to about 900 nm, about 1 nm to about 1,000 nm, about 10 nm to about 50 nm, about 10 nm to about 100 nm, about 10 nm to about 200 nm, about 10 nm to about 300 nm, about 10 nm to about 500 nm, about 10 nm to about 600 nm, about 10 nm to about 700 nm, about 10 nm to about 800 nm, about 10 nm to about 900 nm, about 10 nm to about 1,000 nm, about 50 nm to about 100 nm, about 50 nm to about 200 nm, about 50 nm to about 300 nm, about 50 nm to about 500 nm, about 50 nm to about 600 nm, about 50 nm to about 700 nm, about 50 nm to about 800 nm, about 50 nm to about 900 nm, about 50 nm to about 1,000 nm, about 100 nm to about 200 nm, about 100 nm to about 300 nm, about 100 nm to about 500 nm, about 100 nm to about 600 nm, about 100 nm to about 700 nm, about 100 nm to about 800 nm, about 100 nm to about 900 nm, about 100 nm to about 1,000 nm, about 200 nm to about 300 nm, about 200 nm to about 500 nm, about 200 nm to about 600 nm, about 200 nm to about 700 nm, about 200 nm to about 800 nm, about 200 nm to about 900 nm, about 200 nm to about 1,000 nm, about 300 nm to about 500 nm, about 300 nm to about 600 nm, about 300 nm to about 700 nm, about 300 nm to about 800 nm, about 300 nm to about 900 nm, about 300 nm to about 1,000 nm, about 500 nm to about 600 nm, about 500 nm to about 700 nm, about 500 nm to about 800 nm, about 500 nm to about 900 nm, about 500 nm to about 1,000 nm, about 600 nm to about 700 nm, about 600 nm to about 800 nm, about 600 nm to about 900 nm, about 600 nm to about 1,000 nm, about 700 nm to about 800 nm, about 700 nm to about 900 nm, about 700 nm to about 1,000 nm, about 800 nm to about 900 nm, about 800 nm to about 1,000 nm, or about 900 nm to about 1,000 nm. In some embodiments, the size of nanoparticle ranges from about 1 nm, about 10 nm, about 50 nm, about 100 nm, about 200 nm, about 300 nm, about 500 nm, about 600 nm, about 700 nm, about 800 nm, about 900 nm, or about 1,000 nm. In some embodiments, the size of nanoparticle ranges from at least about 1 nm, about 10 nm, about 50 nm, about 100 nm, about 200 nm, about 300 nm, about 500 nm, about 600 nm, about 700 nm, about 800 nm, or about 900 nm. In some embodiments, the size of nanoparticle ranges from at most about 10 nm, about 50 nm, about 100 nm, about 200 nm, about 300 nm, about 500 nm, about 600 nm, about 700 nm, about 800 nm, about 900 nm, or about 1,000 nm.

In some embodiments, a plurality of nanoparticles includes at least about two nanoparticles. In some embodiments, a plurality of nanoparticles includes at least about three nanoparticles. In some embodiments, a plurality of nanoparticles includes at least about four nanoparticles. In some embodiments, a plurality of nanoparticles includes at least about five nanoparticles. In some embodiments, a plurality of nanoparticles includes at least about six nanoparticles. In some embodiments, a plurality of nanoparticles includes at least about seven nanoparticles. In some embodiments, a plurality of nanoparticles includes at least about eight nanoparticles. In some embodiments, a plurality of nanoparticles includes at least about nine nanoparticles. In some embodiments, a plurality of nanoparticles includes at least about ten nanoparticles.

In some embodiments, disclosed herein is a method of analyzing proteomes on a large scale by increasing throughput several orders of magnitude without compromising identification rates. In some embodiments, an increase of throughput can be achieved by removing the chromatography domain and using nanoparticles for sample complexity reduction maintaining per unit time sample analysis dynamic range to about five orders of magnitude (the linear response range of ESI without ionization suppression).

In some embodiments, disclosed herein is a method of analyzing proteomes on a large scale where increasing instrument resolution can increase the identification rates in a multiplexed environment.

In some embodiments, disclosed herein is a method of analyzing proteomes on a large scale wherein nanoparticle fractionation can increase the identification rate by at least about twice the neat sample.

In some embodiments, disclosed herein is a method of analyzing proteomes on a large scale by direct infusion MS/MS or nLC-MS/MS, at least about 300,000 samples per year per instrument. In some embodiments, disclosed herein is a method of analyzing proteomes on a large scale by direct infusion MS/MS or nLC-MS/MS, at least about 250,000 samples per year per instrument. In some embodiments, disclosed herein is a method of analyzing proteomes on a large scale by direct infusion MS/MS or nLC-MS/MS, at least about 200,000 samples per year per instrument. In some embodiments, disclosed herein is a method of analyzing proteomes on a large scale by direct infusion MS/MS or nLC-MS/MS, at least about 150,000 samples per year per instrument. In some embodiments, disclosed herein is a method of analyzing proteomes on a large scale by direct infusion MS/MS or nLC-MS/MS, at least about 100,000 samples per year per instrument. In some embodiments, disclosed herein is a method of analyzing proteomes on a large scale by direct infusion MS/MS or nLC-MS/MS, at least about 50,000 samples per year per instrument. In some embodiments, disclosed herein is a method of analyzing proteomes on a large scale by direct infusion MS/MS or nLC-MS/MS, at least about 10,000 samples per year per instrument. In some embodiments, disclosed herein is a method of analyzing proteomes on a large scale by direct infusion MS/MS or nLC-MS/MS, at least about 5,000 samples per year per instrument. In some embodiments, disclosed herein is a method of analyzing proteomes on a large scale by direct infusion MS/MS or nLC-MS/MS, at least about 1,000 samples per year per instrument.

In some embodiments, disclosed herein is a method comprising contacting a plurality of biomolecules with a surface to adsorb the plurality of biomolecules on the surface. In some embodiments, disclosed herein is a method comprising contacting a plurality of biomolecules with a plurality of surfaces to adsorb the plurality of biomolecules on the plurality of surfaces. In some embodiments, the method comprises performing mass spectrometry on the plurality of biomolecules to generate a mass spectrum. In some embodiments, the mass spectrometry is performed without chromatographic separation. In some embodiments, the mass spectrometry is performed with direct infusion. In some embodiments, the mass spectrometry is performed using chromatographic separation with a fast gradient length. In some embodiments, the gradient length is at least 30, 25, 20, 15, 10, 9, 8, 7, 6, 5, 4, 3, 2, or 1 minute. In some embodiments, the gradient length is at most 30, 25, 20, 15, 10, 9, 8, 7, 6, 5, 4, 3, 2, or 1 minute. In some embodiments, the gradient length is at least 30, 25, 20, 15, 10, 9, 8, 7, 6, 5, 4, 3, 2, or 1 minute per surface. In some embodiments, the gradient length is at most 30, 25, 20, 15, 10, 9, 8, 7, 6, 5, 4, 3, 2, or 1 minute per surface.

In some embodiments, disclosed herein is a method of analyzing proteomes on a large scale by direct infusion MS/MS or nLC-MS/MS with a sampling rate of about 100%. In some embodiments, disclosed herein is a method of analyzing proteomes on a large scale by direct infusion MS/MS or nLC-MS/MS with a sampling rate of about 95%. In some embodiments, disclosed herein is a method of analyzing proteomes on a large scale by direct infusion MS/MS or nLC-MS/MS with a sampling rate of about 90%. In some embodiments, disclosed herein is a method of analyzing proteomes on a large scale by direct infusion MS/MS or nLC-MS/MS with a sampling rate of about 85%. In some embodiments, disclosed herein is a method of analyzing proteomes on a large scale by direct infusion MS/MS or nLC-MS/MS with a sampling rate of about 80%. In some embodiments, disclosed herein is a method of analyzing proteomes on a large scale by direct infusion MS/MS or nLC-MS/MS with a sampling rate of about 75%. In some embodiments, disclosed herein is a method of analyzing proteomes on a large scale by direct infusion MS/MS or nLC-MS/MS with a sampling rate of about 70%. In some embodiments, disclosed herein is a method of analyzing proteomes on a large scale by direct infusion MS/MS or nLC-MS/MS with a sampling rate of about 65%. In some embodiments, disclosed herein is a method of analyzing proteomes on a large scale by direct infusion MS/MS or nLC-MS/MS with a sampling rate of about 60%. In some embodiments, the sample rate refers to the rate of not missing detectable peptides.

In some embodiment, disclosed herein is a method of analyzing proteomes on a large scale without chromatography separation and by using MS detectors at a scan rate of about 40 Hz. In some embodiment, disclosed herein is a method of analyzing proteomes on a large scale without chromatography separation. In some embodiment, disclosed herein is a method of analyzing proteomes on a large scale by using MS detectors at a scan rate of about 50 Hz. In some embodiments, the scan rate of MS detectors is about 45 Hz. In some embodiments, the scan rate of MS detectors is about 40 Hz. In some embodiments, the scan rate of MS detectors is about 35 Hz. In some embodiments, the scan rate of MS detectors is about 30 Hz.

In some embodiments, the scan rate of MS detectors ranges from about 20 Hz to about 60 Hz. In some embodiments, the scan rate of MS detectors ranges from about 20 Hz to about 25 Hz, about 20 Hz to about 30 Hz, about 20 Hz to about 35 Hz, about 20 Hz to about 40 Hz, about 20 Hz to about 45 Hz, about 20 Hz to about 50 Hz, about 20 Hz to about 55 Hz, about 20 Hz to about 60 Hz, about 25 Hz to about 30 Hz, about 25 Hz to about 35 Hz, about 25 Hz to about 40 Hz, about 25 Hz to about 45 Hz, about 25 Hz to about 50 Hz, about 25 Hz to about 55 Hz, about 25 Hz to about 60 Hz, about 30 Hz to about 35 Hz, about 30 Hz to about 40 Hz, about 30 Hz to about 45 Hz, about 30 Hz to about 50 Hz, about 30 Hz to about 55 Hz, about 30 Hz to about 60 Hz, about 35 Hz to about 40 Hz, about 35 Hz to about 45 Hz, about 35 Hz to about 50 Hz, about 35 Hz to about 55 Hz, about 35 Hz to about 60 Hz, about 40 Hz to about 45 Hz, about 40 Hz to about 50 Hz, about 40 Hz to about 55 Hz, about 40 Hz to about 60 Hz, about 45 Hz to about 50 Hz, about 45 Hz to about 55 Hz, about 45 Hz to about 60 Hz, about 50 Hz to about 55 Hz, about 50 Hz to about 60 Hz, or about 55 Hz to about 60 Hz. In some embodiments, the scan rate of MS detectors ranges from about 20 Hz, about 25 Hz, about 30 Hz, about 35 Hz, about 40 Hz, about 45 Hz, about 50 Hz, about 55 Hz, or about 60 Hz. In some embodiments, the scan rate of MS detectors ranges from at least about 20 Hz, about 25 Hz, about 30 Hz, about 35 Hz, about 40 Hz, about 45 Hz, about 50 Hz, or about 55 Hz. In some embodiments, the scan rate of MS detectors ranges from at most about 25 Hz, about 30 Hz, about 35 Hz, about 40 Hz, about 45 Hz, about 50 Hz, about 55 Hz, or about 60 Hz.

In some embodiments, disclosed herein is a method of analyzing proteomes by using spectral demultiplexing algorithm.

In some embodiments, a machine learning algorithm can be deployed to determine the common characteristics of proteins at different concentration ranges. In some embodiments, for instance, each of the three nanoparticles needs only to resolve three orders of dynamic range which would be within the ESI linear response range without ionization suppression. In some embodiments, if robust ‘concentration-dependent’ nanoparticles are designed, one may only need to inject each sample for 1 minute of MS time. The reasoning is as follows: (1) quadrupole transmission mass range of 400-1600 with a 2 Dalton window scanning at 40 Hz and at 3 separate collision energies can take less than 1 minute ((1200/2)/40×3)=45 seconds per injection (or 2 min per sample); and (2) even 2 Dalton windows can contain multiplexed spectra, and it can be demultiplexed by using linear algebra techniques, such as matrix-based multiple regression, convex optimization, or LU decomposition. Accordingly, in some embodiments, about 80% of capacity per instrument may give over 200 k samples per year per instrument at the upper theoretical limit. In some embodiments, a spectral library can be predicted using tools, such as Prosit.

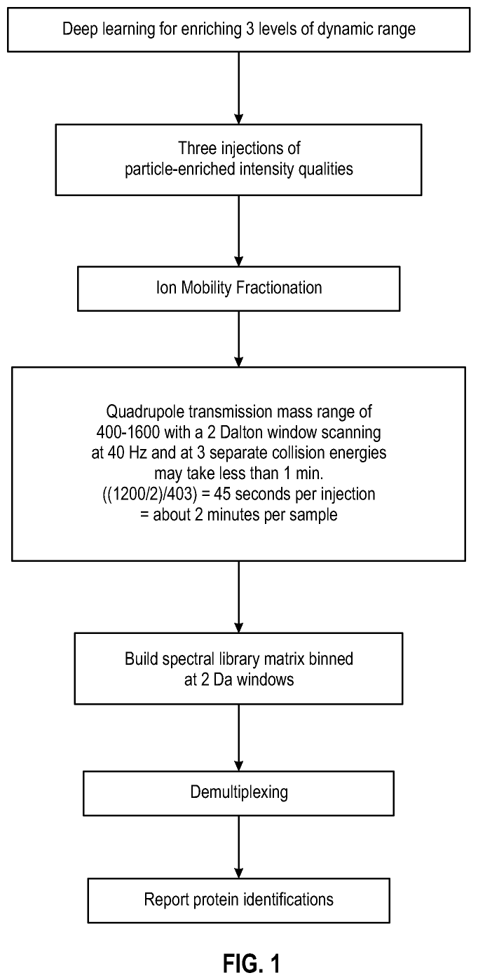

In some embodiments, disclosed herein is a method of identifying proteins including: (1) conducting deep learning for enriching three or more levels of dynamic range; (2) conducting three or more injections of nanoparticle-enriched intensity quartiles (e.g. quadrupole transmission mass range of 400-1600 with a 2 Dalton window scanning rate at 40 Hz and at 3 separate collision energies would take less than 1 min (1200/2)/403)=45 seconds per injection=about 2 minutes per sample); (3) building spectral library matrix binned at 2 Da windows; and (4) applying LU decomposition/window for demultiplexing; and (5) reporting protein identifications (FIG. 1).

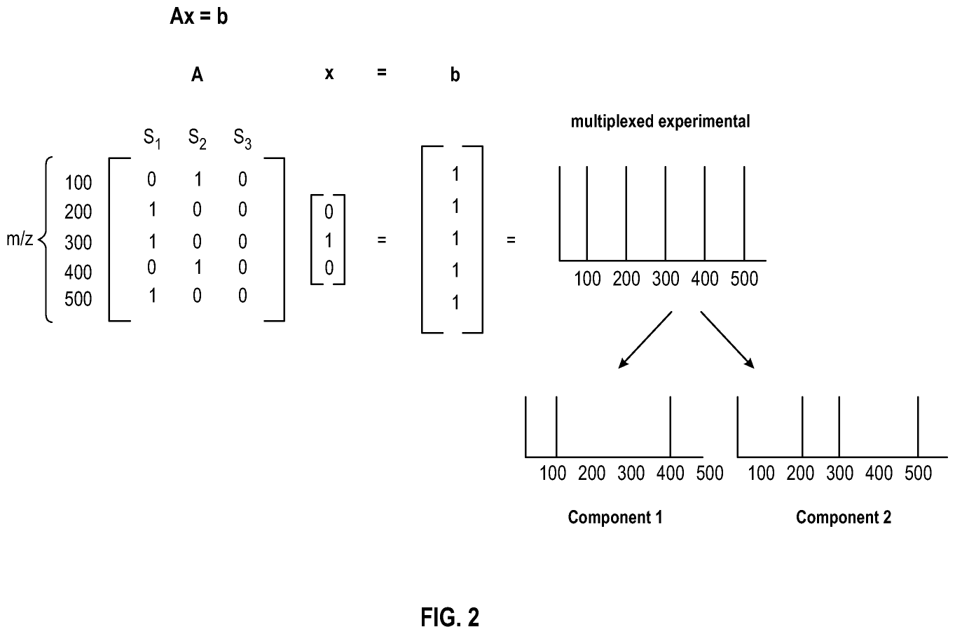

In some embodiments, demultiplexing can be achieved by solving a linear system of equations where a matrix is created for each 2 Da window (FIG. 1). For example, if a precursor bin of 400-402 contains 1000 different theoretical spectra, the matrix may have 1000 columns and N rows (depending on the resolution of the m/z desired). A matrix form of least squares may solve the system where exact solutions are not available. In some embodiments, LU decomposition or convex optimization using non-negativity constraints can also be used. In some embodiments, the example in FIG. 2, the experimental spectrum is multiplex at 1:1 ratio (black/blue). Solving this example may produce two coefficients where ⅔ spectra in the library are detected.

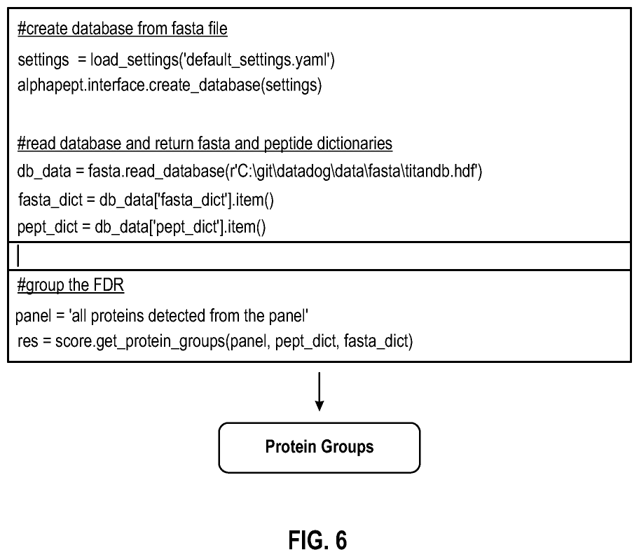

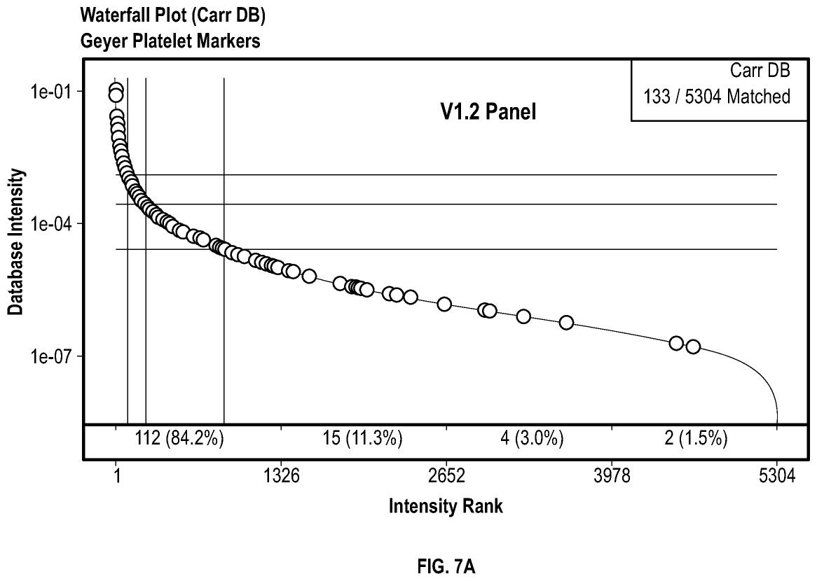

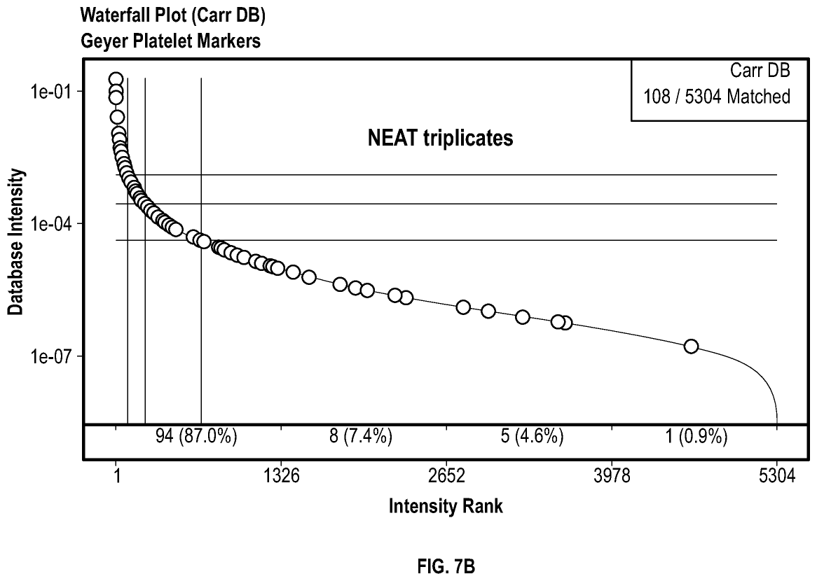

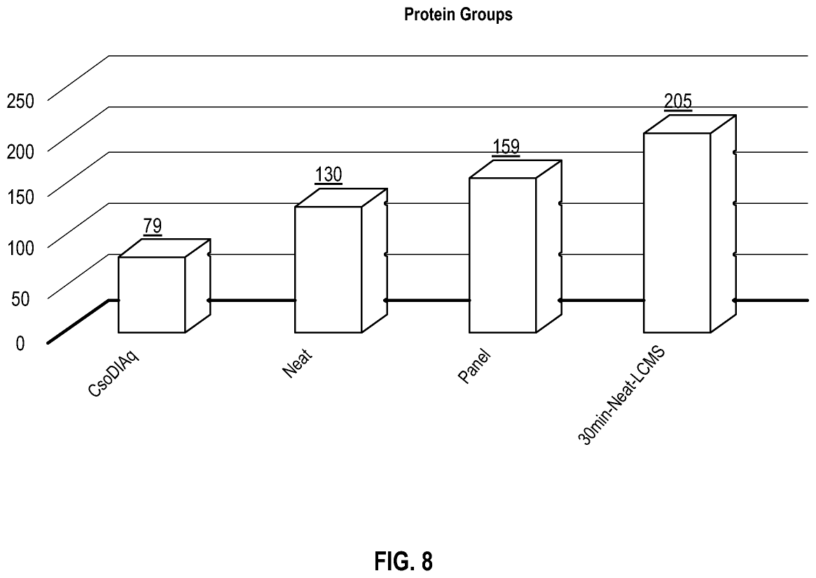

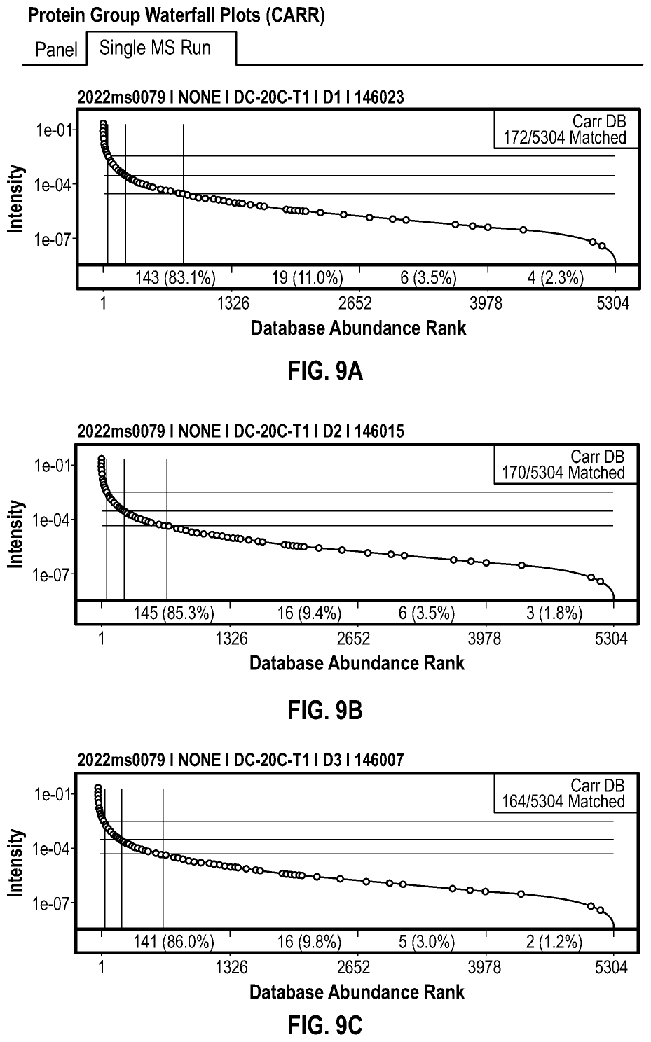

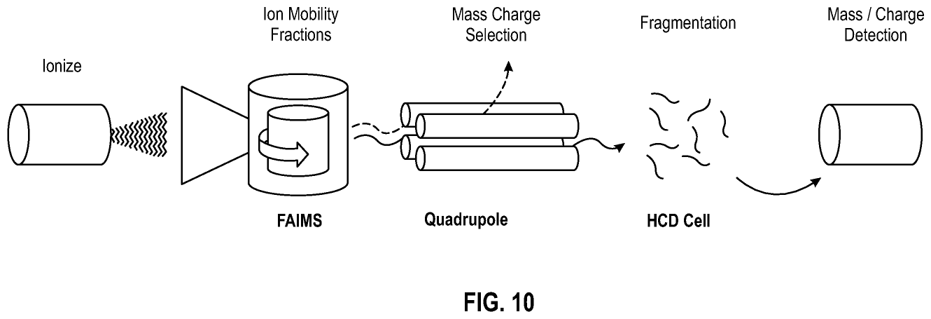

In some embodiments, the number of proteins can be identified by using the Alphapept's “modified” protein inference module, as shown in FIG. 6.

Some embodiments disclosed herein are systems and methods for performing a method that includes processing a biological sample using protein corona formation and analyzing the proteins in said protein corona using direct injection mass spectrometry that does not include chromatographic separation. Methods and systems for performing protein corona formation that can be used in the method are disclosed in U.S. patent application Ser. No. 17/216,520 and U.S. patent application Ser. No. 17/216,523, which are both incorporated herein by reference in their entirety. In some embodiments, protein corona formation occurs on a plurality of distinct surfaces (e.g., particles, such as nanoparticles) with different physiochemical properties that result in different protein coronas, and the different protein coronas are analyzed separately using the direct injection mass spectrometry. In some embodiments, the direct infusion mass spectrometry is performed using electrospray injection followed by ion mobility fractionation. In some embodiments, the electrospray injection for each protein corona analyzed is performed for less than 5 minutes or less than 3 minutes. In some embodiments, the mass spectrometry is tandem mass spectrometry. In some embodiments, the method further includes analyzing the data from the direct injection mass spectrometry to identify proteins in the protein corona. In some embodiments, the analysis includes demultiplexing the data using linear algebra techniques, such as matrix-based multiple regression, convex optimization, or LU decomposition. In some embodiments, the demultiplexing is performed using a machine learning algorithm.

The methods for incubating the composition are not particularly limited, and may be any conditions sufficient to form a protein corona on the one or more surfaces. Methods of forming protein coronas are disclosed in U.S. Pat. No. 11,428,688, which is hereby incorporated by reference in its entirety. In some embodiments, the composition is incubated with the surface at a temperature of at least about 5, 10, 15, 20, 25, 30, 37, 40, 45, 50, 55, 60, 65, 70, 75, 80, 85, 90, or 95° C. In some embodiments, the composition is incubated with the surface at a temperate of at most about 5, 10, 15, 20, 25, 30, 37, 40, 45, 50, 55, 60, 65, 70, 75, 80, 85, 90, or 95° C. In some embodiments, the composition is incubated with the surface at a temperature of about 5° C. to 95° C., 10° C. to 90° C., 15° C. to 85° C., 20° C. to 80° C., 25° C. to 75° C., 30° C. to 70° C., 35° C. to 65° C., 40° C. to 60° C., 45° C. to 55° C., 50° C. to 95° C., or about 37° C. In some embodiments, the composition is incubated with the surface for at least about 5 minutes, 10 minutes, 15 minutes, 20 minutes, 25 minutes, 30 minutes, 35 minutes, 40 minutes, 45 minutes, 50 minutes, 55 minutes, or 1 hour. In some embodiments, the composition is incubated with the surface for at most 5 minutes, 10 minutes, 15 minutes, 20 minutes, 25 minutes, 30 minutes, 35 minutes, 40 minutes, 45 minutes, 50 minutes, 55 minutes, or 1 hour. In some embodiments, the composition is incubated with the surface for at least about 5 minutes to 1 hour, 10 minutes to 55 minutes, 15 minutes to 50 minutes, 20 minutes to 45 minutes, 25 minutes to 40 minutes, or 30 minutes to 35 minutes. In some embodiments, the composition is incubated for at least 15 minutes at a temperate of at least 20° C.

### Biological Sample

In some cases, a biological sample may comprise a cell or be cell-free. In some cases, a biological sample may comprise a biofluid, such as blood, serum, plasma, urine, or cerebrospinal fluid (CSF). In some cases, a biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract. In some cases, a biological sample may be obtained from a subject. In some cases, the subject may be a human or a non-human. In some cases, the subject may be a plant, a fungus, or an archaeon. In some cases, a biological sample can contain a plurality of proteins or proteomic data, which may be analyzed after adsorption or binding of proteins to the one or more surfaces of the various sensor element (e.g., particle) types in a panel and subsequent digestion of protein coronas.

In some cases, a biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some cases, a biological sample may comprise multiple biological samples (e.g., pooled plasma from multiple subjects, or multiple tissue samples from a single subject). In some cases, a biological sample may comprise a single type of biofluid or biomaterial from a single source.

In some cases, a biological sample may be diluted or pre-treated. In some cases, a biological sample may undergo depletion (e.g., the biological sample comprises serum) prior to or following contact with a surface disclosed herein. In some cases, a biological sample may undergo physical (e.g., homogenization or sonication) or chemical treatment prior to or following contact with a surface disclosed herein. In some cases, a biological sample may be diluted prior to or following contact with a surface disclosed herein. In some cases, a dilution medium may comprise buffer or salts, or be purified water (e.g., distilled water). In some cases, a biological sample may be provided in a plurality partitions, wherein each partition may undergo different degrees of dilution. In some cases, a biological sample may comprise may undergo at least about 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution. In some cases, a biological sample may comprise may undergo at most about 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution. In some cases, a biological sample may comprise may undergo about 1.1-fold to 1000-fold, 1.2-fold to 500-fold, 1.3-fold to 200-fold, 1.4-fold to 100-fold, 1.5-fold to 75-fold, 2-fold to 50-fold, 3-fold to 40-fold, 4-fold to 30-fold, 5-fold to 20-fold, 6-fold to 15-fold, 8-fold to 12-fold, or 10-fold to 1000-fold dilution. In some cases, the biological sample may be diluted using a buffer that modified pH of the biological sample. In some case, the pH may be modified to at least about 2, 3, 4, 5, 6, 7, 8, 9, 10. In some case, the pH may be modified to at most about 2, 3, 4, 5, 6, 7, 8, 9, 10. In some case, the pH may be modified to about 2 to 10, 3 to 9, 4 to 8, 5 to 7, 6 to 10. In some case, the pH may be modified to 9-10. In some cases, the pH is modified by dilution with a pH 9.5 Tris buffer. In some cases, the biological sample be separated into portions, and the portions may be adjusted to different pHs before processing using the methods disclosed herein.

In some cases, the biological sample may comprise a plurality of biomolecules. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof. A biological sample may comprise a member of any class of biomolecules, where “classes” may refer to any named category that defines a group of biomolecules having a common characteristic (e.g., proteins, nucleic acids, carbohydrates).

### Proteomic Analysis

As used herein, “proteomic analysis”, “protein analysis”, and the like, may refer to any system or method for analyzing proteins, including glycoproteins, in a sample, including the systems and methods disclosed herein. The present disclosure systems and methods for assaying using one or more surfaces. In some cases, a surface may comprise a surface of a high surface-area material, such as nanoparticles, particles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material). In some cases, the surface may be configured to selectively adsorb extracellular vesicles from a biological sample, For example, the surface may be ammonium-functionalized magnetic particles that may adsorb extra cellular vesicles due, at least in part, to electrostatic interactions. In some cases, the surface may include polyethylene imine functionalized with ammonium. As another example, the surface may include hydrophobic groups (e.g., aromatic groups) and hydrophilic groups (e.g., polyethylene oxide groups) distributed on the surface such that extracellular vesicles selectively adsorb.

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a plasma sample). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least about 5, 6, 7, 8, 9, 10, 15, or 20 magnitudes. In some cases, a panel including any number of distinct particle types disclosed herein, may enrich and identify biomolecules over a dynamic range of at most about 5, 6, 7, 8, 9, 10, 15, or 20 magnitudes. In some cases, a panel including any number of distinct particle types disclosed herein, may enrich and identify biomolecules over a dynamic range of about 5 to 20, 6 to 15, 7 to 10, or 8 to 9 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a particle coated with an outer layer of silica and a particle coated with an outer layer of poly(dimethyl aminopropyl methacrylamide) (PDMAPMA). A particle panel consistent with the present disclosure could also comprise two or more particles selected from the group consisting of silica coated particle, an N-(3-Trimethoxysilylpropyl)diethylenetriamine coated particle, a PDMAPMA coated particle, a carboxyl-functionalized polyacrylic acid coated particle, an amino surface functionalized particle, a polystyrene carboxyl functionalized particle, and a dextran coated particle. A particle panel consistent with the present disclosure may also comprise two or more particles selected from the group consisting of a surfactant free carboxylate functionalized particle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-saccharide functionalized polystyrene particles, amine functionalized polystyrene particles, polystyrene carboxyl functionalized particles, ubiquitin functionalized polystyrene particles, dextran coated particles, or any combination thereof. In some cases, the particle panel may comprise silica-coated particles, amine-functionalized particles, amine-functionalized polymer-coated particles, and carboxylate-functionalized particles.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxy silylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxy silylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group, a carbamoyl group, a hydroxyl group, an aldehyde group and the like. In some cases, a polar functional group may comprise a primary amine group, a secondary amine group, a tertiary amine group, a quaternary amine group, a cyclic secondary amine group, a primary amide group, a secondary amide group, a tertiary amide group, an imine group, a pyridyl group, a pyrimidine group, a pyrrolidinium group, an imidazole group, a guanidine group, a guanidinium group, or any combination thereof.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle. In some cases, a positively charged particle may have a zeta potential of more than about 0, 5, 10, 15, 20, 25, 50, or 100 mV. In some cases, a negative charged particle may have a zeta potential of less than about 0, −5, −10, −15, −20, −25, −50, or −100 mV. In some cases, a positively charged particle may have a zeta potential of more than about 0 to 100, 5 to 50, 10 to 25, or 15 to 20 mV.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may comprise a protein, polynucleotide, or polysaccharide, or may be comparable in size to any of the aforementioned classes of species. In some cases, a surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a surface functionalization may comprise pKa of about 1 to 14, 2 to 13, 3 to 12, 4 to 11, 5 to 10, 6 to 9, or 7 to 8. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle, or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. In some cases, a linker may be about 0.5 to 30, 1 to 25, 2 to 20, 3 to 15, 4 to 9, 5 to 8, or 6 to 7 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique glycoproteins in a biological sample. In some cases, a method may identify at least about 1, 2, 3, 4, 5, 6, 7, 8, 9, 10, 20, 30, 40, 50, 60, 70, 80, 90, 100, or 200 unique glycoproteins. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique glycoproteins. In some cases, a method may identify about 1 to 10000, 2 to 9000, 3 to 8000, 4 to 7000, 5 to 6000, 6 to 5000, 7 to 4000, 8 to 3000, 9 to 2000, 10 to 1000, 20 to 900, 30 to 800, 40 to 700, 50 to 600, 60 to 500, 70 to 400, 80 to 300, 90 to 200, or 100 to 10000 unique glycoproteins.

In some cases, a method of the present disclosure may identify a large number of unique biomolecules (e.g., proteins) in a biological sample (e.g., a biofluid). In some cases, a surface disclosed herein may be incubated with a biological sample to adsorb at least about 1000, 2000, 3000, 4000, 5000, 6000, 7000, 8000, 9000, or 10000 unique biomolecules. In some cases, a surface disclosed herein may be incubated with a biological sample to adsorb at most about 1000, 2000, 3000, 4000, 5000, 6000, 7000, 8000, 9000, or 10000 unique biomolecules. In some cases, a surface disclosed herein may be incubated with a biological sample to adsorb at least about 1000 to 10000, 2000 to 9000, 3000 to 8000, 4000 to 7000, or 5000 to 6000 unique biomolecules. In some cases, a surface disclosed herein may be incubated with a biological sample to adsorb at least about 1000, 2000, 3000, 4000, 5000, 6000, 7000, 8000, 9000, or 10000 unique biomolecule groups. In some cases, a surface disclosed herein may be incubated with a biological sample to adsorb at most about 1000, 2000, 3000, 4000, 5000, 6000, 7000, 8000, 9000, or 10000 unique biomolecule groups. In some cases, a surface disclosed herein may be incubated with a biological sample to adsorb about 1000 to 10000, 2000 to 9000, 3000 to 8000, 4000 to 7000, or 5000 to 6000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify about 1 to 10000, 2 to 9000, 3 to 8000, 4 to 7000, 5 to 6000, 6 to 5000, 7 to 4000, 8 to 3000, 9 to 2000, 10 to 1000, 20 to 900, 30 to 800, 40 to 700, 50 to 600, 60 to 500, 70 to 400, 80 to 300, 90 to 200, or 100 to 10000 unique proteoforms. In some cases, a surface disclosed herein may be incubated with a biological sample to adsorb at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb about 1 to 10000, 2 to 9000, 3 to 8000, 4 to 7000, 5 to 6000, 6 to 5000, 7 to 4000, 8 to 3000, 9 to 2000, 10 to 1000, 20 to 900, 30 to 800, 40 to 700, 50 to 600, 60 to 500, 70 to 400, 80 to 300, 90 to 200, or 100 to 10000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, the panels disclosed herein can be used to identify a number of proteins, peptides, protein groups, or protein classes using a protein analysis workflow described herein (e.g., a protein corona analysis workflow). In some cases, the panels disclosed herein can be used to identify at least about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at most about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify about 100 to 100000, 200 to 90000, 300 to 80000, 400 to 70000, 500 to 60000, 600 to 50000, 700 to 40000, 800 to 30000, 900 to 20000, 1000 to 10000, 2000 to 9000, 3000 to 8000, 4000 to 7000, or 5000 to 6000 unique proteins. In some cases, the panels disclosed herein can be used to identify at least about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at most about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify about 100 to 100000, 200 to 90000, 300 to 80000, 400 to 70000, 500 to 60000, 600 to 50000, 700 to 40000, 800 to 30000, 900 to 20000, 1000 to 10000, 2000 to 9000, 3000 to 8000, 4000 to 7000, or 5000 to 6000 protein groups. In some cases, the panels disclosed herein can be used to identify at least about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, the panels disclosed herein can be used to identify at most about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, the panels disclosed herein can be used to identify about 100 to 100000, 200 to 90000, 300 to 80000, 400 to 70000, 500 to 60000, 600 to 50000, 700 to 40000, 800 to 30000, 900 to 20000, 1000 to 10000, 2000 to 9000, 3000 to 8000, 4000 to 7000, or 5000 to 6000 peptides.

In some cases, a peptide may be a tryptic peptide. In some cases, a peptide may be a semi-tryptic peptide. In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least about 2 proteins, 5 proteins, 10 proteins, 20 proteins, 40 proteins, 60 proteins, 80 proteins, 100 proteins, 150 proteins, 200 proteins, or more. In some cases, a protein class may contain at most about 2 proteins, 5 proteins, 10 proteins, 20 proteins, 40 proteins, 60 proteins, 80 proteins, 100 proteins, 150 proteins, 200 proteins, or more. In some cases, a protein class may contain about 2 proteins to 200 proteins, 5 proteins to 150 proteins, 10 proteins to 100 proteins, 20 proteins to 80 proteins, 40 proteins to 60 proteins, or more.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, and other protein separation techniques.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papain, Asp N, thermolysine, pepsin, aspartyl protease, cathepsin D, zinc metalloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of about 8 to 15 amino acids. In some cases, the digestion may generate an average peptide fragment length of about 12 to 18 amino acids. In some cases, the digestion may generate an average peptide fragment length of about 15 to 25 amino acids. In some cases, the digestion may generate an average peptide fragment length of about 20 to 30 amino acids. In some cases, the digestion may generate an average peptide fragment length of about 30 to 50 amino acids. In some cases, the digestion may generate an average peptide fragment length of at least about 8, 10, 15, 20, 25, 30, 35, 40, 45, 50 amino acids. In some cases, the digestion may generate an average peptide fragment length of at most about 8, 10, 15, 20, 25, 30, 35, 40, 45, 50 amino acids.

In some cases, proteins or peptides may be prepared for mass spectrometry. In some cases, proteins or peptides may be treated with an alkylating agent. For example, proteins or peptides may be treated with N-ethylmaleimide and iodoacetamide. In some case, proteins or peptides may be treated with a reducing agent. For example, proteins or peptides are treated with dithiothreitol (DTT) or tris(2-carboxyethyl)phosphine (TCEP). In some cases, proteins or peptides are digested, alkylated, and reduced before analysis. In some cases, proteins or peptides are digested, alkylated, and reduced before analysis using mass spectrometry.

In some cases, an assay may rapidly generate and analyze proteomic data. In some cases, beginning with an input biological sample (e.g., a buccal or nasal smear, plasma, or tissue), a method of the present disclosure may generate and analyze proteomic data in less than about 1, 2, 3, 4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. The particles disclosed herein can have surface area to mass ratios of 3 to 30 cm 2/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

In some cases, a particle may comprise a nanoparticle. In some cases, a particle may comprise a microparticle. In some cases, a first particle, a second particle, or both particles in a particle panel are nanoparticles. In some cases, a first particle, a second particle, or both particles in a particle panel are microparticles. In some cases, a first particle may be a nanoparticle and a second particle may be a microparticle.

In some cases, a particle may comprise a diameter of at least about 10, 20, 30, 40, 50, 60, 70, 80, 90, 100, 200, 300, 400, 500, 600, 700, 800, 900, or 1000 nm. In some cases, a particle may comprise a diameter of at most about 10, 20, 30, 40, 50, 60, 70, 80, 90, 100, 200, 300, 400, 500, 600, 700, 800, 900, or 1000 nm. In some cases, a particle may comprise a diameter of about 10 to 1000, 20 to 900, 30 to 800, 40 to 700, 50 to 600, 60 to 500, 70 to 400, 80 to 300, 90 to 200, or 100 to 1000 nm. In some cases, a particle may comprise a diameter of at least about 10, 20, 30, 40, 50, 60, 70, 80, 90, 100, 200, 300, 400, 500, 600, 700, 800, 900, or 1000 μm. In some cases, a particle may comprise a diameter of at most about 10, 20, 30, 40, 50, 60, 70, 80, 90, 100, 200, 300, 400, 500, 600, 700, 800, 900, or 1000 μm. In some cases, a particle may comprise a diameter of about 10 to 1000, 20 to 900, 30 to 800, 40 to 700, 50 to 600, 60 to 500, 70 to 400, 80 to 300, 90 to 200, or 100 to 1000 μm.

In some cases, a first size of a first particle is at least about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1000 times a second size of a second particle. In some cases, a first size of a first particle is at most about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1000 times a second size of a second particle. In some cases, a first size of a first particle is about 1.1 to 1000, 1.2 to 900, 1.3 to 800, 1.4 to 700, 1.5 to 600, 1.6 to 500, 1.7 to 400, 1.8 to 300, 1.9 to 200, 2 to 100, 3 to 90, 4 to 80, 5 to 70, 10 to 60, 20 to 50, or 30 to 40 times a second size of a second particle. In some cases, a size of a first particle is within ±40% of a size of a second particle, a size of a first particle is within ±30% of a size of a second particle, a size of a first particle is within ±25% of a size of a second particle, a size of a first particle is within ±20% of a size of a second particle, a size of a first particle is within ±15% of a size of a second particle, or a size of a first particle is within ±10% of a size of a second particle. In some cases, a size of a first particle is within at least about ±10%, ±15%, ±20%, ±25%, ±30%, ±35%, ±40% of a size of a second particle. In some cases, a size of a first particle is within at most about ±10%, ±15%, ±20%, ±25%, ±30%, ±35%, ±40% of a size of a second particle. In some cases, a size of a first particle is within about ±10% to ±40%, ±15% to ±35%, ±20% to ±30%, ±25% to ±40% of a size of a second particle. In some cases, the first size is a first diameter, and the second size is a second diameter. In some cases, the first size is a first average size, and the second size is a second average size. In some cases, the first average size and the second average size are mean sizes or median sizes.

In some cases, a particle panel comprises at least about 1, 2, 3, 4, 5, 6, 7, 8, 9, or 10 parts of a first particle to about 1 part of a second particle. In some cases, a particle panel comprises at most about 1, 2, 3, 4, 5, 6, 7, 8, 9, or 10 parts of a first particle to about 1 part of a second particle. In some cases, a particle panel comprises about 1 to 10, 2 to 9, 3 to 8, 4 to 7, 5 to 6 parts of a first particle to about 1 part of a second particle. In some cases, a particle panel comprises about 15 parts of a first particle to about 6 parts of a second particle. In some cases, the parts are parts by weight, parts by volume, or parts by surface area. In some cases, the parts are parts by weight.

### Non-Specific Binding

A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least about 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 fg/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 pg/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 ng/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 μg/mm². In some cases, biomolecules or proteins may adsorb at a density of about 0.1 to 1000, 0.2 to 900, 0.3 to 800, 0.4 to 700, 0.5 to 600, 0.6 to 500, 0.7 to 400, 0.8 to 300, 0.9 to 200, 1 to 100, 2 to 90, 3 to 80, 4 to 70, 5 to 60, 6 to 50, 7 to 40, 8 to 30, 9 to 20, or 10 to 1000 mg/mm².

Adsorbed biomolecules may comprise various types of proteins. In some cases, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise about 1 to 10000, 2 to 9000, 3 to 8000, 4 to 7000, 5 to 6000, 6 to 5000, 7 to 4000, 8 to 3000, 9 to 2000, 10 to 1000, 20 to 900, 30 to 800, 40 to 700, 50 to 600, 60 to 500, 70 to 400, 80 to 300, 90 to 200, 100 to 10000 types of proteins.

In some cases, proteins in a biological sample may comprise at least about 1, 2, 3, 4, 5, 6, 7, 8, 9, 10, 15, 20, 25, or 30 orders of magnitudes in concentration. In some cases, proteins in a biological sample may comprise at most about 1, 2, 3, 4, 5, 6, 7, 8, 9, 10, 15, 20, 25, or 30 orders of magnitudes in concentration. In some cases, proteins in a biological sample may comprise about 1 to 30, 2 to 25, 3 to 20, 4 to 15, 5 to 10, 6 to 9, or 7 to 8 orders of magnitudes in concentration.

### Classification Using Machine Learning

In some cases, identifications of biomolecules using the method disclosed herein may be processed using a machine learning algorithm. In some cases, the identifications of biomolecules may comprise identifications of nucleic acids, variants thereof, proteins, variants thereof, and any combination thereof. In some cases, the machine learning algorithm may be an unsupervised or self-supervised learning algorithm. In some cases, the machine learning algorithm may be trained to learn a latent representation of the identifications of the biomolecules. In some cases, the machine learning algorithm may be supervised learning algorithm. In some cases, the machine learning algorithm may be trained to learn to associate a given set of identifications with a value associated with a predetermined task. For example, the predetermined task may comprise determining a disease state associated with the given set of identifications, where the value may indicate the probability of the disease state being present in a subject associated with the given set of identifications.

In some cases, the method of determining a set of biomolecules associated with the disease or disorder and/or disease state can include the analysis of the biomolecule corona of at least two samples. This determination, analysis or statistical classification can be performed by methods, including, but not limited to, for example, a wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive Bayes, linear regression, polynomial regression, SVM for regression, K-means clustering, and hidden Markov models, among others. In other words, the biomolecules in the corona of each sample are compared/analyzed with each other to determine with statistical significance what patterns are common between the individual corona to determine a set of biomolecules that is associated with the disease or disorder or disease state.

In some cases, machine learning algorithms can be used to construct models that accurately assign class labels to examples based on the input features that describe the example. In some case it may be advantageous to employ machine learning and/or deep learning approaches for the methods described herein. For example, machine learning can be used to associate the biomolecule corona with various disease states (e.g. no disease, precursor to a disease, having early or late stage of the disease, etc.). For example, in some cases, one or more machine learning algorithms can be employed in connection with the methods disclosed hereinto analyze data detected and obtained by the biomolecule corona and sets of biomolecules derived therefrom. For example, machine learning can be coupled with genomic and proteomic information obtained using the methods described herein to determine not only if a subject has a pre-stage of cancer, cancer or does not have or develop cancer, and also to distinguish the type of cancer.

In some cases, machine learning algorithms may also be used to associate the results from protein corona analysis and results from nucleic acid sequencing analysis and further associate any trends or correlations between proteins and nucleic acids to a biological state (e.g., disease state, health state, subtypes of disease such as stages of disease are cancer subtypes).

In some cases, machine learning may be used to cluster proteins detected using a plurality of surfaces. In some cases, a panel of surfaces may be used to assay proteins from one or more biological samples. In some cases, a surface in the panel of surfaces may comprise diverse physicochemical properties. In some cases, proteins detected by the panel of surfaces may be clustered using a clustering algorithm. In some cases, proteins detected by the panel of surfaces may be clustered based at least partially on the intensities of detected protein signals, particle chemical properties, protein structural and/or functional groups, or any combination thereof.

A panel of surfaces may comprise any number of surfaces. In some cases, a panel of surfaces may comprise at least about 1, 2, 3, 4, 5, 6, 7, 8, 9, 10, 20, 30, 40, 50, 60, 70, 80, 90, 100, 200, 300, 400, 500, 600, 700, 800, 900, or 1000 surfaces. In some cases, a panel of surfaces may comprise at most about 1, 2, 3, 4, 5, 6, 7, 8, 9, 10, 20, 30, 40, 50, 60, 70, 80, 90, 100, 200, 300, 400, 500, 600, 700, 800, 900, or 1000 surfaces.

Inputs to a machine learning algorithm may comprise various kinds of inputs. In some cases, an input may comprise a value that represents a physicochemical property of a surface used to assay a biomolecule. A physicochemical property of a particle may comprise various properties disclosed herein, which includes: charge, hydrophobicity, hydrophilicity, amphipathicity, coordinating, reaction class, surface free energy, various functional groups/modifications (e.g., sugar, polymer, amine, amide, epoxy, crosslinker, hydroxyl, aromatic, or phosphate groups). In some cases, an input may comprise a value that represents a parameter of a given assay. A parameter may comprise incubation conditions including temperature, incubation time, pH, buffer type, and any variables in performing an assay disclosed herein.

In some cases, a clustering algorithm can refer to a method of grouping samples in a dataset by some measure of similarity. In some cases, samples can be grouped in a set space, for example, element ‘a’ is in set ‘A’. In some cases, samples can be grouped in a continuous space, for example, element ‘a’ is a point in Euclidean space with distance ‘l’ away from the centroid of elements comprising cluster ‘A’. In some cases, samples can be grouped in a graph space, for example, element ‘a’ is highly connected to elements comprising cluster ‘A’. In some cases, clustering can refer to the principle of organizing a plurality of elements into groups in some mathematical space based on some measure of similarity.

In some cases, clustering can comprise grouping any number of biomolecules in a dataset by any quantitative measure of similarity. In some cases, clustering can comprise K-means clustering. In some cases, clustering can comprise hierarchical clustering. In some cases, clustering can comprise using random forest models. In some cases, clustering can comprise boosted tree models. In some cases, clustering can comprise using support vector machines. In some cases, clustering can comprise calculating one or more N−1 dimensional surfaces in N-dimensional space that partitions a dataset into clusters. In some cases, clustering can comprise distribution-based clustering. In some cases, clustering can comprise fitting a plurality of prior distributions over the data distributed in N-dimensional space. In some cases, clustering can comprise using density-based clustering. In some cases, clustering can comprise using fuzzy clustering. In some cases, clustering can comprise computing probability values of a data point belonging to a cluster. In some cases, clustering can comprise using constraints. In some cases, clustering can comprise using supervised learning. In some embodiments, clustering can comprise using unsupervised learning.

In some cases, clustering can comprise grouping biomolecules based on similarity. In some cases, clustering can comprise grouping biomolecules based on quantitative similarity. In some cases, clustering can comprise grouping biomolecules based on one or more features of each protein. In some cases, clustering can comprise grouping biomolecules based on one or more labels of each protein. In some cases, clustering can comprise grouping biomolecules based on Euclidean coordinates in a numerical representation of biomolecules. In some cases, clustering can comprise grouping biomolecules based on protein structural groups or functional groups (e.g., protein structures, substructures, or functional groups from protein databases such as Protein Data Bank or CATH Protein Structure Classification database). In some cases, a protein structural group or functional group may comprise protein primary structure, secondary structure, tertiary structure, or quaternary structure. In some cases, a protein structural group or functional group may be based at least partially on alpha helices, beta sheets, relative distribution of amino acids with different properties (e.g., aliphatic, aromatic, hydrophilic, acidic, basic, etc.), a structural families (e.g., TIM barrel and beta barrel fold), protein domains (e.g., Death effector domain). In some cases, a protein structural group or functional group may be based at least partially on functional or spatial properties (e.g., functional groups-group of immune globulins, cytokines, cytoskeletal biomolecules, etc.).

### Definitions

As used herein, the term “biomolecule corona” generally refers to the plurality of different biomolecule that bind to a surface. The term “protein corona” generally refers to proteins and optionally other plasma components that bind to surfaces (e.g., nanoparticles) when they come into contact with biological samples or a biological system. For use herein, the term “protein corona” also encompasses both the soft and hard protein corona as referred to in Milani et al. “Reversible versus Irreversible Binding of Transferring to Polystyrene Nanoparticles: Soft and Hard Corona” ACS NANO, 2012, 6(3), pp. 2532-2541; Mirshafiee et al. “Impact of protein pre-coating on the protein corona composition and nanoparticle cellular uptake” Biomaterials vol. 75, January 2016 pp. 295-304, Mahmoudi et al. “Emerging understanding of the protein corona at the nano-bio interfaces” Nanotoday 11(6) December 2016, pp. 817-832, and Mahmoudi et al. “Protein-Nanoparticle Interactions: Opportunities and Challenges” Chem. Rev., 2011, 111(9), pp. 5610-5637, the contents of which are incorporated by reference in their entireties. As described therein, an adsorption curve may show the build-up of a strongly bound monolayer up to the point of monolayer saturation (at a geometrically defined protein-to-NP ratio), beyond which a secondary, weakly bound layer is formed. While the first layer is irreversibly bound (hard corona), the secondary layer (soft corona) may exhibit dynamic exchange. Proteins that adsorb with high affinity may form the “hard” corona, comprising tightly bound proteins that do not readily desorb, and proteins that adsorb with low affinity may form the “soft” corona, comprising loosely bound proteins. Soft and hard corona can also be characterized based on their exchange times. Hard corona may show much larger exchange times in the order of several hours. See, e.g., M. Rahman et al. Protein-Nanoparticle Interactions, Spring Series in Biophysics 15, 2013, incorporated by reference in its entirety.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1. Direct Infusion MS/MS Analysis of Extracted Four Proteomes

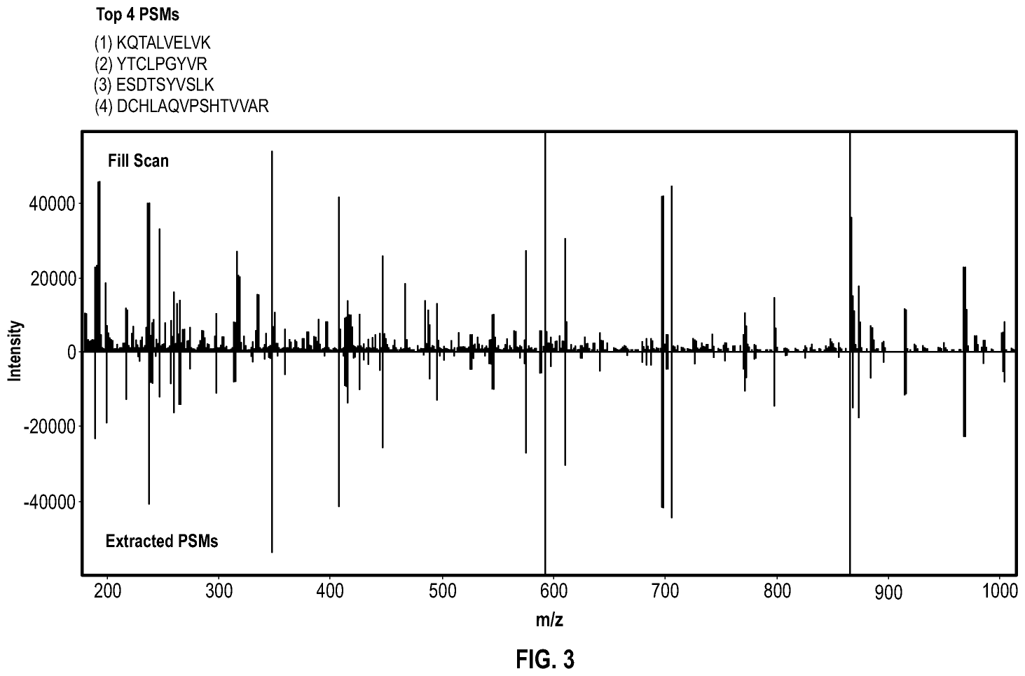

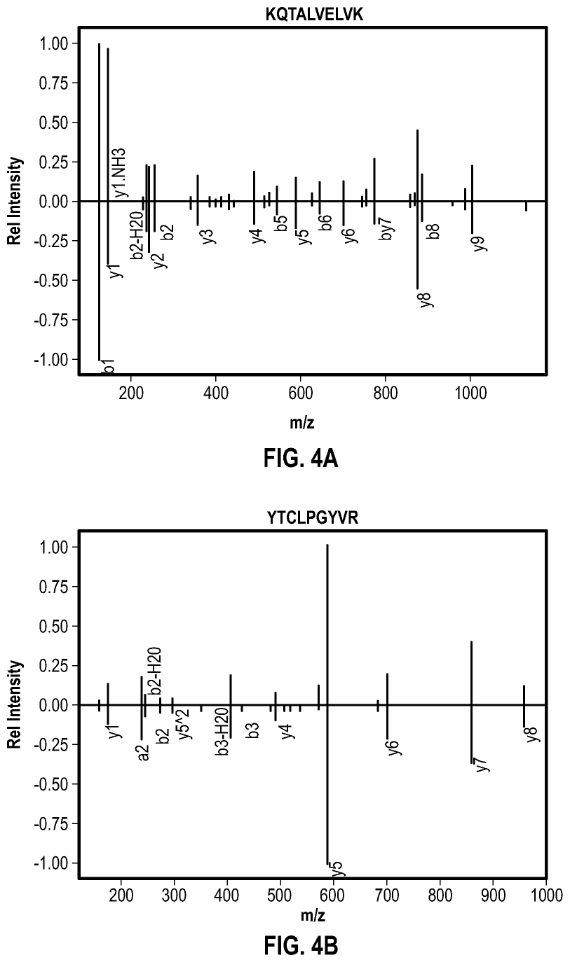

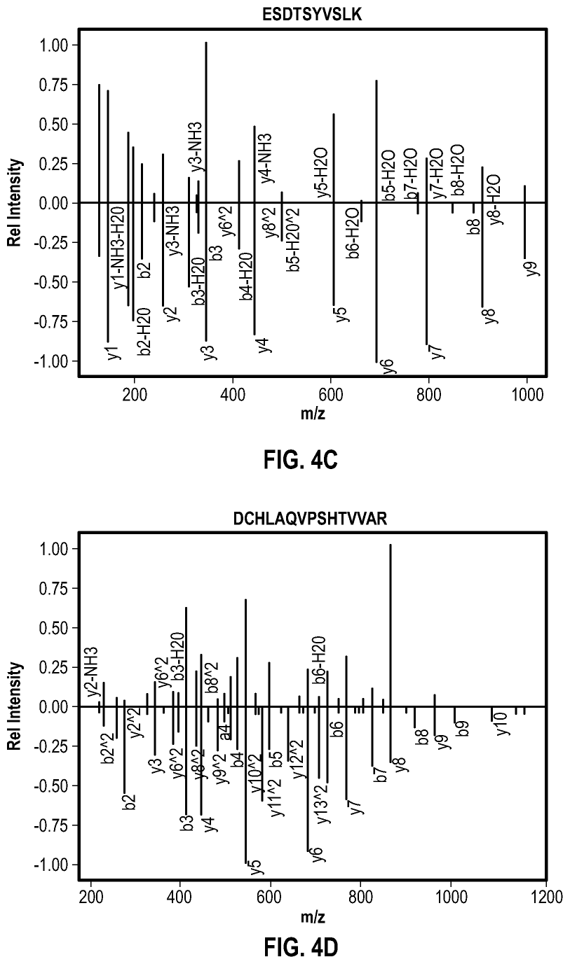

The extracted four proteomes ((1) KQTALVELVK (SEQ ID NO: 1); (2) YTCLPGYVR (SEQ ID NO: 2); (3) ESDTSYVSLK (SEQ ID NO: 3); and (4) DCHLAQVPSHTVVAR (SEQ ID NO: 4)) were analyzed by direct infusion MS/MS. (FIG. 3 and FIGS. 4A-4D).

### Example 2. Finding Unique Peptides by Using Nanoparticles and Direct Infusion at Different Resolutions

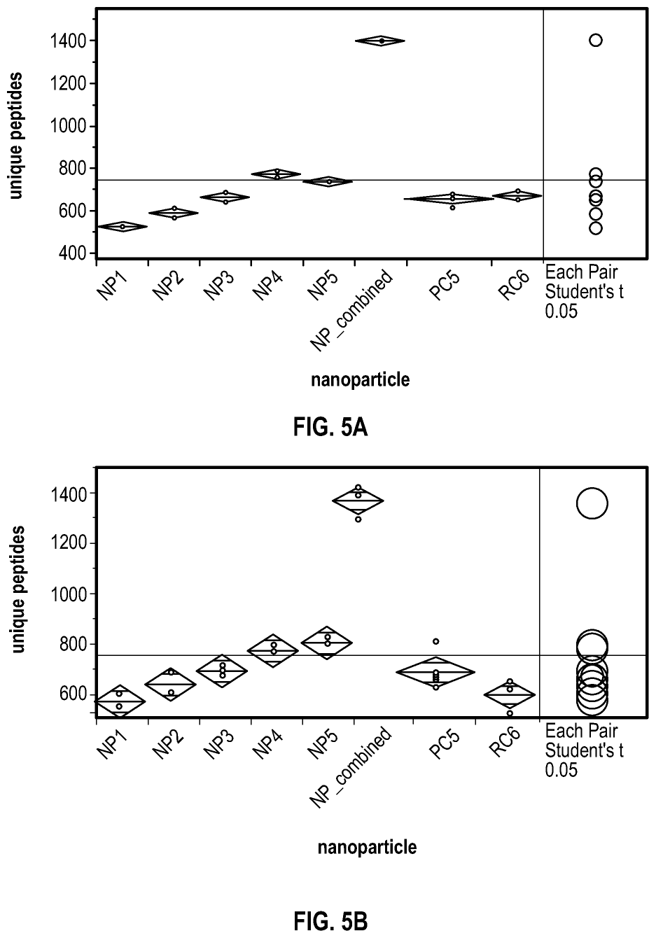

Unique peptides were found by using direct infusion nanoparticles at 60 k resolution (FIG. 5A and Table 1) and at 120 k resolution (FIG. 5B and Table 2).

TABLE 1
Number of Unique Peptides Found by Nanoparticles
at 60k (Means for One-Way ANOVA; Std. error
uses a pooled estimate of error variance.)

| | | | | Lower | Upper |
|---|---|---|---|---|---|
| Level | Number | Mean | Std. error | 95% | 95% |
| NP1 | 3 | 523.00 | 12.263 | 497.2 | 548.8 |
| NP2 | 3 | 586.33 | 12.263 | 560.6 | 612.1 |
| NP3 | 3 | 660.00 | 12.263 | 634.2 | 685.8 |
| NP4 | 3 | 765.67 | 12.263 | 739.9 | 791.4 |
| NP5 | 3 | 735.00 | 12.263 | 709.2 | 760.8 |
| NP combined | 3 | 1403.00 | 12.263 | 1377.2 | 1428.8 |
| PC5 (neat | 5 | 649.80 | 9.499 | 629.8 | 669.8 |
| plasma | | | | | |
| control) | | | | | |
| RC6 (pooled | 3 | 667.33 | 12.263 | 641.6 | 693.1 |
| peptide | | | | | |
| control) | | | | | |

TABLE 2
Number of Unique Peptides Found by Nanoparticles
at 120k (Means for Oneway Anova; Std. error
uses a pooled estimate of error variance.)

| | | | | Lower | Upper |
|---|---|---|---|---|---|
| Level | Number | Mean | Std. error | 95% | 95% |
| NP1 | 3 | 569.00 | 28.936 | 508.2 | 629.8 |
| NP2 | 3 | 631.67 | 28.936 | 570.9 | 692.5 |
| NP3 | 3 | 692.33 | 28.936 | 631.5 | 753.1 |
| NP4 | 3 | 771.33 | 28.936 | 710.5 | 832.1 |
| NP5 | 3 | 791.67 | 28.936 | 730.9 | 852.5 |
| NP combined | 3 | 1357.00 | 28.936 | 1296.2 | 1417.8 |
| PC5 (neat | 5 | 674.60 | 22.413 | 627.5 | 721.7 |
| plasma | | | | | |
| control) | | | | | |
| RC6 (pooled | 3 | 598.67 | 28.936 | 537.9 | 659.5 |
| peptide | | | | | |
| control) | | | | | |

TABLE 3
Conditions for Experimental Matrices

| Total mass spec | | | | | |
|---|---|---|---|---|---|
| time (min) | Max injection time | 25 ms | Mass range | 300 | 1200 |

| | | | | | | | |
|---|---|---|---|---|---|---|---|
| For all 3 CVs | | | | | | | |
| Resolution/Isolation | 25 | 4 | 2 | 1 | 0.7 | Resolution | Transience |
| window | | | | | | | length |
| | | | | | | | (ms) |
| 240k | 0.92 | 5.76 | 11.52 | 23.04 | 32.91 | 240k | 512 |
| 120k | 0.46 | 2.88 | 5.76 | 11.52 | 16.46 | 120k | 256 |
| 60k | 0.23 | 1.44 | 2.88 | 5.76 | 8.23 | 60k | 128 |
| 30k | 0.12 | 0.72 | 1.44 | 2.88 | 4.11 | 30k | 64 |
| 15k | 0.06 | 0.36 | 0.72 | 1.44 | 2.06 | 15k | 32 |
| 7.5k | 0.05 | 0.28 | 0.56 | 1.13 | 1.61 | 7.5k | 25 |

### Example 3. Protein Analysis of Plasma Sample without Chromatographic Separation

A plasma sample is processed using the commercially-available PROTEOGRAPH Assay Kit (v 1.2). Five samples of peptides (one for each nanoparticle) are obtained, and each peptide sample is analyzed by direct injection mass spectrometry (MS-MS with ion mobility fractionation; 2 minute ESI injection time). The resulting mass spectrometry data is demultiplexed using linear algebra techniques, such as matrix-based multiple regression, convex optimization, or LU decomposition, to identify protein groups within the plasma sample. By analyzing the peptide samples without chromatographic separation, this example can provide a significant reduction in mass spectrometry run time per sample.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A method comprising:
(a) contacting a plurality of biomolecules with a surface to adsorb the plurality of biomolecules on the surface; and
(b) performing mass spectrometry on the plurality of biomolecules to generate a mass spectrum, wherein the mass spectrometry is (i) performed without chromatographic separation or (ii) performed using chromatographic separation with a gradient length of at most 30 minutes.

2. The method of claim 1, wherein the gradient length is at most 25, 20, 15, 10, 9, 8, 7, 6, 5, 4, 3, 2, or 1 minute.

3. The method of claim 1, wherein the chromatographic separation uses a chromatography column comprising a length of at most 30, 25, 20, 15, 10, 5, 4, 3, 2, 1, 0.9, 0.8, 0.7, 0.6, 0.5, 0.4, 0.3, 0.2, or 0.1 centimeters.

4. The method of claim 1, wherein the performing the mass spectrometry comprises performing ion mobility separation.

5. The method of claim 4, wherein the ion mobility separation is performed using travelling-wave ion mobility separation.

6. The method of claim 5, wherein the travelling-wave ion mobility separation is performed along a distance greater than 5 m or greater than 10 m.

7. The method of claim 5 or 6, wherein the travelling-wave ion mobility separation is performed along a serpentine path.

8. The method of claim 4, wherein the ion mobility separation is performed using high field asymmetric waveform ion mobility spectrometry (FAIMS).

9. The method of claim 1 or 4-8, wherein the performing the mass spectrometry comprises performing direct infusion mass spectrometry.

10. The method of claim 9, wherein the direct infusion mass spectrometry is performed in less than about 10 minutes, less than about 5 minutes, less than about 3 minutes, or less than about 2 minutes.

11. The method of any one of claims 1-10, wherein the mass spectrometry is performed with an isolation width of about 0.4, 0.6, 0.8, 1, or 2 m/z.

12. The method of any one of claims 1-11, wherein the mass spectrometry is performed with a maximum injection time of about 500 ms, 1 s, 5 s, 30 s, 1 min, 2 min, 3 min, 4 min, 5 min, or 10 min.

13. The method of any one of claims 1-12, wherein the mass spectrometry is performed with a resolution of about 120, 240, or 500 K.

14. The method of any one of claims 1-13, wherein the mass spectrometry is performed with a resolution of about 30K to about 800K.

15. The method of any one of claims 1-14, wherein the contacting the plurality of biomolecules with the surface to adsorb the plurality of biomolecules on the surface comprises contacting a biological sample with the surface.

16. The method of claim 15, wherein the biological sample comprises blood, plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

17. The method of claim 15, wherein the biological sample comprises blood, serum, or plasma.

18. The method of any one of claims 15-17, wherein the biological sample is a biofluid.

19. The method of claim 15, wherein the biological sample comprises cell lysate.

20. The method of any one of claims 1-18, wherein the surface is a particle.

21. The method of any one of claims 1-20, wherein the contacting the plurality of biomolecules with the surface to adsorb the plurality of biomolecules on the surface comprises incubating the biomolecules with the surface for at least 15 minutes.

22. The method of claim 21, wherein the incubating is performed at a temperature of about 5° C. to about 40° C.

23. The method of any one of claims 1-22, wherein the mass spectrometry comprises tandem mass spectrometry.

24. The method of any one of claims 1-23, wherein the mass spectrometry comprises Fourier Transform mass spectrometry.

25. The method of any one of claims 1-24, wherein the mass spectrometry comprises time-of-flight mass spectrometry.

26. The method of any one of claims 1-25, wherein said performing mass spectrometry identifies at least about 300 protein groups.

27. The method of any one of claims 1-26, wherein the performing mass spectrometry comprises data independent acquisition (DIA).

28. The method of any one of claims 1-27, wherein the surface comprises a plurality of particles having a polydispersity index of greater than 0.5, greater than 0.75, or greater than 1.0.

29. The method of any one of claims 1-27, wherein the surface comprises a plurality of particles having a polydispersity index of less than 0.1.

30. The method of any one of claims 1-29, wherein the plurality of biomolecules comprise a plurality of proteins.

31. The method of any one of claims 1-30, wherein the surface selectively adsorbs extracellular vesicles.

32. The method of claim 31, wherein the surface is ammonium-functionalized.

33. The method of claim 32, wherein the surface comprises polyethyleneimine.

34. The method of claim 32, wherein the surface comprises crosslinked polyethyleneimine.

35. The method of claim 31, wherein the surface comprises polyethylene oxide and an aromatic group.

36. A method comprising:
(a) contacting a plurality of biomolecules with a surface to adsorb the plurality of biomolecules on the surface;
(b) performing mass spectrometry on the plurality of biomolecules to generate a multiplexed mass spectrum; and
(c) deconvoluting the multiplexed mass spectrum to generate a plurality of deconvoluted MS signals, wherein the deconvoluting is based on a type of the surface.

37. The method of claim 36, wherein the deconvoluting is based on genomic information associated with the plurality of biomolecules.

38. The method of claim 37, wherein the genomic information comprises a spectral library of a plurality of expressible polyamino acids by an organism.

39. The method of any one of claims 36-38, wherein the spectral library comprises a plurality of mass spectral signals of the plurality of expressible polyamino acids.

40. The method of any one of claims 36-39, wherein the performing the mass spectrometry comprises scanning the plurality of biomolecules across a plurality of mass windows.

41. The method of claim 40, wherein the scanning is performed at a frequency between 20 and 80 Hertz.

42. The method of any one of claims 40-41, wherein the plurality of mass windows is between 400 and 1600 Daltons.

43. The method of any one of claims 40-42, wherein the plurality of mass windows comprises a window of about 2 Daltons.

44. The method of any one of claims 40-43, wherein the multiplexed mass spectrum comprises signals within the window.

45. The method of any one of claims 40-44, wherein the plurality of mass spectral signals in the spectral library is within the window.

46. The method of any one of claims 36-45, wherein the deconvoluting is based on a spectral library of a subset of the plurality of expressible polyamino acids, wherein the subset is configured to be adsorbed preferentially to the surface compared to an expressible polyamino acid not in the subset.

47. The method of any one of claims 36-46, wherein the plurality of biomolecules is less abundant compared to the most abundant biomolecule in a sample by at most about 6 orders of magnitude.

48. The method of any one of claims 36-47, wherein the plurality of biomolecules is less abundant compared to the most abundant biomolecule in a sample by at least about 5 orders of magnitude and at most about 8 orders of magnitude.

49. The method of any one of claims 36-48, wherein the plurality of biomolecules is less abundant compared to the most abundant biomolecule in a sample by at least about 7 orders of magnitude.

50. The method of any one of claims 36-49, wherein the contacting the plurality of biomolecules with the surface to adsorb the plurality of biomolecules on the surface comprises contacting a biological sample with the surface.

51. The method of claim 50, wherein the biological sample comprises blood, plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

52. The method of claim 50, wherein the biological sample comprises blood, serum, or plasma.

53. The method of claim 50, wherein the biological sample comprises cell lysate.

54. The method of any one of claims 50-52, wherein the biological sample is a biofluid.

55. The method of any one of claims 36-54, wherein the surface is a particle.

56. The method of any one of claims 36-55, wherein the contacting the plurality of biomolecules with the surface to adsorb the plurality of biomolecules on the surface comprises incubating the biomolecules with the surface for at least 15 minutes.

57. The method of claim 56, wherein the incubating is performed at a temperature of about 5° C. to about 40° C.

58. The method of any one of claims 36-57, wherein the mass spectrometry comprises tandem mass spectrometry.

59. The method of any one of claims 36-58, wherein the mass spectrometry comprises Fourier Transform mass spectrometry.

60. The method of any one of claims 36-59, wherein the mass spectrometry comprises time-of-flight mass spectrometry.

61. The method of any one of claims 36-60, wherein the mass spectrometry comprises data independent acquisition (DIA).

62. The method of any one of claims 36-61, wherein the mass spectrometry comprises a targeted mass spectrometry.

63. The method of claim 62, wherein the targeted mass spectrometry comprises multiple reaction monitoring (MRM).

64. The method of any one of claims 36-63, wherein the surface selectively adsorbs extracellular vesicles.

65. The method of claim 64, wherein the surface is ammonium-functionalized.

66. The method of claim 65, wherein the surface comprises polyethyleneimine.

67. The method of claim 65, wherein the surface comprises crosslinked polyethyleneimine.

68. The method of claim 64, wherein the surface comprises polyethylene oxide and an aromatic group.

69. A method comprising:
(a) incubating a biological sample with one or more surfaces to form a biomolecule corona on the one or more surfaces; and
(b) performing mass spectrometry on biomolecules from the biomolecule corona to generate a mass spectrum, wherein the mass spectrometry is (i) performed without chromatographic separation or (ii) performed using chromatographic separation with a gradient length of at most 10 minutes.

70. The method of claim 69, wherein the gradient length is at most 9, 8, 7, 6, 5, 4, 3, 2, or 1 minute.

71. The method of claim 69 or 70, wherein the chromatographic separation uses a chromatography column comprising a length of at most 30, 25, 20, 15, 10, 5, 4, 3, 2, 1, 0.9, 0.8, 0.7, 0.6, 0.5, 0.4, 0.3, 0.2, or 0.1 centimeters.

72. The method of claim 69, wherein the performing the mass spectrometry comprises performing ion mobility separation.

73. The method of claim 72, wherein the ion mobility separation is performed using high field asymmetric waveform ion mobility spectrometry (FAIMS).

74. The method of claim 72, wherein the ion mobility separation is performed using travelling-wave ion mobility separation.

75. The method of claim 74, wherein the travelling-wave ion mobility separation is performed along a distance greater than 5 m or greater than 10 m.

76. The method of claim 74 or 75, wherein the travelling-wave ion mobility separation is performed along a serpentine path.

77. The method of any one of claims 69-72, wherein the performing mass spectrometry comprises performing direct infusion mass spectrometry.

78. The method of any one of claims 69-77, wherein the performing mass spectrometry is without chromatographic separation.

79. The method of any one of claims 69-78, wherein the performing mass spectrometry comprises tandem mass spectrometry.

80. The method of any one of claims 69-79, wherein the performing mass spectrometry comprises data independent acquisition (DIA).

81. The method of any one of claims 69-79, wherein the performing mass spectrometry comprises a targeted mass spectrometry.

82. The method of claim 81, wherein the targeted mass spectrometry comprises multiple reaction monitoring (MRM).

83. The method of any one of claims 69-82, wherein the performing mass spectrometry comprises electrospray injection.

84. The method of any one of claims 69-83, the biomolecules from the biomolecule corona comprise proteins.

85. The method of claim 84, wherein the performing mass spectrometry comprises detecting at least 50, 100, 200, 250, 300, 400, 500, or 1000 proteins in the biological sample.

86. The method of claim 84, wherein the performing mass spectrometry comprises detecting at least 50, 100, 200, 250, 300, 400, 500, or 1000 protein groups in the biological sample.

87. The method of any one of claims 69-86, further comprising, before (b), removing biomolecules from the biomolecule corona.

88. The method of claim 87, wherein the removing biomolecules from biomolecule corona comprises digesting the biomolecule corona with an enzyme.

89. The method of claim 88, wherein the enzyme is a protease, such as trypsin.

90. The method of any one of claims 69-86, further comprising, before (b), alkylating and reducing the biomolecules from the biomolecule corona.

91. The method of any one of claims 69-87, further comprising, before (b), alkylating, reducing, and digesting the biomolecules from the biomolecule corona.

92. The method of any one of claims 69-91 further comprising, before (b), apply one or more washing steps to the biomolecules from the biomolecule corona.

93. The method of any one of claims 69-92, wherein the incubating comprises maintaining the biological sample at a temperature of 20° C. to 50° C. for at least 15 minutes.

94. The method of any one of claims 69-92, wherein the biological sample comprises blood, plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

95. The method of any one of claims 69-94, wherein the biological sample comprises blood, serum, or plasma.

96. The method of any one of claims 69-92, wherein the biological sample comprises cell lysate.

97. The method of any one of claims 69-95, wherein the one or more surfaces comprise a particle.

98. The method of claim 97, wherein the particle is a porous particle.

99. The method of claim 97 or 98, wherein the particle is a microparticle.

100. The method of claim 97 or 98, wherein the particle is nanoparticle.

101. The method of any one of claims 97-100, wherein the particle comprises a paramagnetic material.

102. The method of claim 101, wherein the paramagnetic material is a superparamagnetic material.

103. The method of claim 101 or 102, wherein the paramagnetic material comprises iron oxide

104. The method of any one of claims 69-103, wherein the biological sample is incubated with a first surface and a second surface having different physicochemical properties such that different protein coronas are formed on each surface.

105. The method of claim 104, wherein the first surface and the second surface comprise a different sign of charge.

106. The method of claim 104, wherein the first surface and the second surface comprise a different sign of zeta potential.

107. The method of claim 104, wherein the first surface and the second surface comprise the same sign of charge.

108. The method of claim 104, wherein the first surface and the second surface comprise the same sign of zeta potential.

109. The method of any one of claims 104-108, wherein the first surface, the second surface, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof.

110. The method of any one of claims 104-109, wherein the first surface and the second surface are incubated in separate aliquots of the biological sample.

111. The method of claim 110, wherein, in (b), mass spectrometry is performed separately for the biomolecule corona from the first surface and the biomolecule corona from the second surface.

112. The method of any one of claims 104-109, wherein the first surface and the second surface are incubated in a single volume of the biological sample.

113. The method of any one of claims 69-112, further comprising, before (b), washing the biomolecule corona with a wash composition.

114. The method of any one of claims 69-113, further comprising determining a biological state of a subject using the mass spectrum.

115. The method of any one of claims 69-114, wherein the biological sample is incubated with at least 2, 3, 4, 5, 6, 7, 8, 9, or 10 surfaces having different physicochemical properties.

116. The method of any one of claims 69-114, where the biological sample is incubated with 3 to 8 surfaces having different physicochemical properties.

117. The method of claim 115 or 116, wherein the performing mass spectrometry comprises at least 2, 3, 4, 5, 6, 7, 8, 9, or 10 separate injections.

118. The method of claim 115 or 116, wherein the performing mass spectrometry comprises 3-8 separate injections.

119. The method of claim 117 or 118, wherein each of the separate injections correspond to biomolecule corona obtained from different surfaces.

120. The method of any one of claims 69-119, wherein at least one of the surfaces selectively adsorbs extracellular vesicles.

121. The method of claim 120, wherein the at least one of the surfaces is ammonium-functionalized.

122. The method of claim 121, wherein the at least one of the surfaces comprise polyethyleneimine.

123. The method of claim 121, wherein the at least one of the surfaces comprise crosslinked polyethyleneimine.

124. The method of claim 120, wherein the at least one of the surfaces comprises polyethylene oxide and an aromatic group.

125. A method comprising:
(a) incubating a biological sample with a first magnetic particle to form a first protein corona;
(b) incubating the biological sample with a second magnetic particle to form a second protein corona, wherein the first magnetic particle and the second magnetic particle have different physicochemical properties, and wherein the first magnetic particle and the second magnetic particle are incubated with separate volumes of the biological sample; and
(c) performing mass spectrometry using direct infusion on proteins from the first protein corona and the second protein corona to generate a mass spectrum, wherein separate injections are performed for the first protein corona and the second protein corona.

126. The method of claim 125, wherein at least 500 different protein groups are detected.

127. The method of claim 125, wherein at least 1000 different protein groups are detected.

128. The method of any one of claims 125-127, wherein the biological sample is blood, serum, or plasma.

129. The method of any one of claims 125-128, wherein the first magnetic particle and the second magnetic particle have a different sign of zeta potential.

130. The method of any one of claims 125-129, where an outer surface of the first magnetic particle comprises an amine functional group and an outer surface of the second magnetic particle comprises a carboxyl functional group.

131. The method of any one of claims 125-130, wherein the first magnetic particle comprises a polymeric outer surface and the second magnetic particle comprises a non-polymeric outer surface.

132. The method of any one of claims 125-131, wherein at least 50 unique protein groups are detected from the first protein corona that are not detected from the second protein corona

133. The method of any one of claims 125-131, wherein at least 100 unique protein groups are detected from the first protein corona that are not detected from the second protein corona.

134. The method of any one of claims 125-133, wherein the first magnetic particle and the second magnetic particle each have a polydispersity index less than 0.25.

135. The method of any one of claims 125-133, wherein the first magnetic particle and the second magnetic particle each have a polydispersity index less than 0.1.

136. The method of any one of claims 125-135, wherein the first magnetic particle and the second magnetic particle are nanoparticles.

137. The method of any one of claims 125-136, wherein the first magnetic particle and the second magnetic particle have a diameter less than 500 nm.

138. The method of any one of claims 125-137, wherein the mass spectrometry is performed in less than 10 minutes.


SEQUENCE LISTING (4 sequences)

SEQ ID NO: 1 - protein, 10 aa, synthetic construct
KQTALVELVK

SEQ ID NO: 2 - protein, 9 aa, synthetic construct
YTCLPGYVR

SEQ ID NO: 3 - protein, 10 aa, synthetic construct
ESDTSYVSLK

SEQ ID NO: 4 - protein, 15 aa, synthetic construct
DCHLAQVPSHTVVAR